(19) United States
(12) Patent Application Publication
(10) Pub. No.: US 2013/0172631 A1
(43) Pub. Date: Jul. 4, 2013

(54) Process for Monitoring Separation of Ethanol Mixture

(71) Applicant: CELANESE INTERNATIONAL CORPORATION, Irving, TX (US)

(72) Inventors: R. Jay Warner, Houston, TX (US); Victor J. Johnston, Houston, TX (US)

(73) Assignee: CELANESE INTERNATIONAL CORPORATION, Irving, TX (US)

(21) Appl. No.: 13/716,893

(22) Filed: Dec. 17, 2012

Related U.S. Application Data
(60) Provisional application No. 61/576,709, filed on Dec. 16, 2011.
(60) Provisional application No. 61/576,715, filed on Dec. 16, 2011.
(60) Provisional application No. 61/576,726, filed on Dec. 16, 2011.

(51) Int. Cl. C07C 29/149

(52) U.S. Cl. CPC C07C 29/149
USPC 568/884

(57) ABSTRACT

A process is disclosed for monitoring separation streams of an ethanol purification process, including measuring the concentrations of impurities, monitoring one or more binary streams, or monitoring conductivity in an ethanol containing stream to determine acetic acid concentrations. The ethanol is produced by hydrogenating acetic acid. One or more on-line analyzers may be used to monitor the separation streams.

### CROSS REFERENCE TO RELATED APPLICATIONS

The present invention claims priority to U.S. Provisional App. Nos. 61/576,709; 61/576,715; and 61/576,726, each filed Dec. 16, 2011. The entire contents and disclosures of the above cited applications are hereby incorporated by reference.

### FIELD OF THE INVENTION

The present invention relates generally to a method of improving monitoring of alcohol purification using analyzers, and, in particular, to a method of monitoring purification of a crude ethanol product obtained by hydrogenating acetic acid. The invention further relates to methods for determining various properties and concentrations of streams in the purification process.

### BACKGROUND OF THE INVENTION

Chemical manufacturing processes typically operate in the liquid or gas phase within a set of operating conditions, such as temperature, pressure, reactant feed rates, reactant concentrations, and catalyst concentration, to yield a crude mixture from which a material having a desired set of physical and chemical properties may be obtained. Modern chemical manufacturing processes typically employ computer-based control to maintain product quality in the final material. In the production of vinyl acetate the analyzers may be used to measure concentrations of various compounds to adjust the feed to the reactor as described in U.S. Pat. No. 6,420,595. In the production of acetic acid, methyl acetate concentration may be measured to adjust a reactor condition as described in U.S. Pat. No. 6,677,480.

Ethanol for industrial use is conventionally produced from petrochemical feed stocks, such as oil, natural gas, or coal, from feed stock intermediates, such as syngas, or from starchy materials or cellulose materials, such as corn or sugar cane. Fermentation processes may use computer-based control systems to monitor the reaction conditions to allow maximum yeast growth and distillation conditions for removing stillage, as described in U.S. Pub. Nos. 2008/0167852, 2008/0109200, and 2008/0103748.

In addition to these conventional production routes, there are also developments in producing ethanol by the reduction of alkanoic acids and/or other carbonyl group-containing compounds. This process produces an aqueous mixture that contains ethanol and several organic components. A challenge for producing ethanol in this manner is that the organic component concentration may be outside of industrial ethanol standards and thus requires separation of the aqueous mixture.

Due to the difference in production and organic impurities, the computer-based control systems for fermentation processes would provide little utility for ethanol production from acetic acid hydrogenation.

Therefore, what is needed is a monitor suitable for controlling the organic components when separating ethanol from the aqueous mixture.

### SUMMARY OF THE INVENTION

In one embodiment, the invention is directed to a process for producing ethanol comprising hydrogenating an acetic acid feed stream in the presence of a catalyst to Balm a crude ethanol product; separating the crude ethanol product in one or more distillation columns, wherein acetic acid is present in a residue and ethanol is present in a distillate, and wherein at least one of the distillate or residue comprises an organic impurity; measuring temperature, pressure, density, concentration, or conductivity of at least one of the distillate or the residue from the one or more distillation columns; setting a baseline value for the at least one of the distillate or the residue; adjusting at least one column parameter based on the measured data and the baseline value; and recovering the ethanol product.

In another embodiment, the invention is directed to a process for producing ethanol, comprising hydrogenating an acetic acid feed stream in the presence of a catalyst to form a crude ethanol product; separating at least a portion of the crude ethanol product in a first column into a first distillate comprising ethanol, water, and ethyl acetate, and a first residue comprising a first binary stream; separating at least a portion of the first distillate in a second column into a second distillate comprising ethyl acetate, and a second residue comprising ethanol, water, and at least one organic impurity; separating at least a portion of the second residue in a third column into a third distillate comprising a second binary stream and a third residue comprising a third binary stream; measuring data of at least one of the distillate or residue to determine data that includes at least one temperature, pressure, density, concentration, or conductivity; setting a baseline value for the one or more measurements; adjusting at least one column parameter based on the measured data and the baseline value; and recovering the ethanol product.

In a second embodiment, the present invention is directed to a process for effecting process control in a distillation column for separating a reaction mixture produced by hydrogenating acetic acid to form ethanol, wherein the reaction mixture comprises ethanol and acetic acid, the process comprising the steps of measuring a conductivity of a condensed distillate of the distillation column, wherein the condensed distillate comprises ethanol and acetic acid; controlling at least one parameter of the distillation column in response to the measured conductivity; setting a baseline conductivity value for the condensed distillate and comparing the measured conductivity with the baseline conductivity value; returning the condensed distillate to the distillation column based on the measured conductivity; and recovering ethanol from the condensed distillate.

In a third embodiment, the present invention is directed to a process for producing ethanol, comprising hydrogenating an acetic acid feed stream in the presence of a catalyst to form a crude ethanol product; separating at least a portion of the crude ethanol product in a first column into a first distillate comprising ethanol, water, and ethyl acetate, and a first residue comprising acetic acid; separating at least a portion of the first distillate in a second column into a second distillate comprising ethyl acetate, and a second residue comprising ethanol, water, and at least one organic impurity; separating at least a portion of the second residue in a third column into a third distillate comprising ethanol and a third residue comprising water; measuring the concentration of the at least one organic impurity using one or more on-line analyzers; and adjusting at least one column parameter of the second column to maintain the organic impurity concentration below 1 wt. %.

### BRIEF DESCRIPTION OF DRAWINGS

The invention is described in detail below with reference to the appended drawings, wherein like numerals designate similar parts.

FIG. 1 is a schematic diagram of an analyzer for a distillation column in accordance with one embodiment of the present invention.

FIG. 2 is a graph of conductivity responses of a binary mixture and a non-aqueous mixture.

FIG. 3 is a graph of acetic acid concentration and conductivity.

FIG. 4 is a graph comparing actual conductivity versus calculated conductivity.

FIG. 5 is a schematic diagram of hydrogenation process having one or more distillation columns in accordance with one embodiment of the present invention.

FIG. 6 is a schematic diagram of hydrogenation process having one or more distillation columns and water separation units in accordance with one embodiment of the present invention.

FIG. 7 is a schematic diagram of hydrogenation process having a conductivity sensor in the condensed distillate of the second column in accordance with one embodiment of the present invention.

### DETAILED DESCRIPTION OF THE INVENTION

The present invention relates to processes for monitoring and controlling separation conditions during the production of ethanol from hydrogenating acetic acid. The hydrogenation reactor produces an ethanol mixture that contains ethanol and water. In some embodiments, the ethanol mixture may also contain impurities that are formed including but not limited to ethyl acetate, acetaldehyde, acetone, diethyl ether, isopropanol, n-propanol, n-butanol, and/or diethyl acetal. Another impurity present in the crude ethanol product is acetic acid that is not converted during the hydrogenation. When the ethanol mixture is separated in one or more separation units the resulting streams may comprise impurities. To obtain ethanol having an industrial acceptance impurity level, it is preferable to remove the impurities. Embodiments of the present invention provide a process control to establish a baseline by (1) using one or more on-line analyzers to measure at least one impurity in any stream of any column in the purification process or (2) inferring the concentration of the impurities by measuring variables of streams. The parameters of the column may be modified or adjusted based on the information, e.g., baseline, from the on-line analyzer or the inferred concentration.

In one embodiment, the present invention utilizes on-line analyzers to infer the concentrations of impurities in streams that comprise mostly of two compounds, which is referred to herein as “binary streams.” In another embodiment, the present invention may utilize on-line analyzers to measure the conductivity of a condensed distillate stream or portion thereof, to determine the impurity concentration. When in large qualities, due to lower conversions, acetic acid is generally removed in the first distillation column. Other impurities are light compounds and have lower boiling points than ethanol. These impurities are preferably removed together in one light ends column during the separation of the ethanol mixture. Subsequent to removing the impurities, water may be separated from the ethanol. Any impurities that are not removed in the light ends column remain with the ethanol and concentrate in the ethanol product. The concentration of impurities contaminants the ethanol product and may render the ethanol unsuitable for its intended application.

Preferably, the on-line analyzer measures the concentration of at least one impurity in a residue stream of a light ends column and adjusts at least one column parameter of the light ends column in response to the impurity concentration. An unacceptable impurity level may vary depending on the ethanol application, but generally any impurity concentration that is greater than 1000 wppm is unacceptable for most ethanol applications. Embodiments of the present invention control the process by maintaining each impurity within an acceptable range. The acceptable ranges may vary for each impurity. An acceptable range for ethyl acetate, n-propanol, or isopropanol may be from 0.1 wppm to 500 wppm, e.g., from 5 to 250 wppm, or from 10 to 100 wppm. An acceptable range for acetaldehyde, diethyl acetal, diethyl ether, n-butanol and C₄-C₅ alcohols may be from 0.1 wppm to 250 wppm, e.g., from 0.5 to 150 wppm, or from 1 to 20 wppm. Although the on-line analyzer may detect each impurity, it may be sufficient to control the process by measuring at least one of the impurities. For example, it may be sufficient to monitor the concentration of isopropanol since this impurity tends to co-distill with ethanol.

To control the process in response to an impurity level, the system may adjust at least one column parameter. Preferably the system adjusts a column parameter of the light ends column. The column parameters include adjusting the reflux ratio, residue to feed ratio, distillate to feed ratio, column temperature, column pressure, reboiler energy input, and combination thereof. The process may also have varying levels of response to different impurity measurements. This may avoid large disruptions to plant operations that result in shutdown and start up costs as well as loss of ethanol product due to smaller impurity changes. The type of response may be determined by the plant operators and adjusted as necessary. A total system shutdown may be necessary when the impurity level exceeds 1 wt. % in the residue stream. In some embodiment rather than a total system shutdown there may be a total reflux, while increasing the reflux ratio may be necessary when the impurity concentration of one compound is above 50 wppm. Audible and/or visual alerts may signal disruptions in impurity concentrations.

The on-line analyzers suitable for use in the present invention include gas chromatograph, high-performance liquid chromatograph (HPLC), mass spectrometer, and infrared or near-infrared spectrometer. Preferably, the impurity concentration may be measured using a gas chromatograph analyzer, an infrared spectrometer, or Raman spectrometer. The measurement may be made in real-time or in near-real-time to provide feedback to the column to control the impurity concentration within an acceptable limit.

The information obtained from the on-line analyzer may be used in combination with another process control measurement such as a conductivity value, pH value, total carbon, and/or content. In addition, the on-line analyzer may be used in combination with information inferred from binary stream compositions. The binary streams may comprise acetic acid and water, or ethanol and water.

In some embodiments, the process control may also control the amount of impurities in an ethyl acetate co-product. In the light ends column, ethyl acetate may also be recovered as a co-product. Controlling the impurity in the ethyl acetate co-product is also necessary to produce a useful commercial product. In the light ends column, the light organics, which includes ethyl acetate and impurities, are preferably removed overhead. The ethyl acetate may be further recovered as a residue stream in a subsequent column. The process control may adjust the subsequent column to regulate the amount of impurities in the ethyl acetate co-product. The ethyl acetate co-product preferably has less than 1 wt. % of impurities, which include acetaldehyde, acetone, diethyl ether, isopropanol, n-propanol, n-butanol, and/or diethyl acetal. The ethyl acetate co-product may comprise ethanol and/or water. In particular it is necessary to control acetaldehyde concentration in the ethyl acetate co-product.

### Binary Streams

As stated above, on-line analyzers may be used to measure a binary stream comprises two compounds. Preferably, total concentration of those two compounds are greater than 90 wt. %, e.g., greater than 95 wt. % or greater than 99 wt. %. The binary streams comprise acetic acid and water, or ethanol and water. Any other compound in the binary stream may be present in minor concentrations and do not have a substantial impact on the composition. In other words, the binary streams of the present invention are treated as binary based on the primary compounds, but this does not exclude the presence of other compounds.

To monitor the separation of the crude ethanol product, embodiments of the present invention monitor the composition of the binary stream without actually measuring the concentration of the stream. In one embodiment, the present invention may use information, including pressure, temperature, and/or density, in a separation process to infer the composition of the binary stream. Because these streams comprise of two major components, the inferred concentrations using measured pressure, temperature, and/or density provides real-time inferred composition data with high accuracy in comparison to the actual composition of the stream. The real-time inferred composition data may be further used to control one or more column and/or reactor parameters.

In accordance to an embodiment, the composition may be based on information obtained by measuring the temperature, pressure, and/or density of a binary stream. Determining the binary stream composition by involve using density measured, pressure-compensated temperature information, or temperature-compensated density information to infer the composition of the binary stream. More than one of these methods may be used in combination to infer the composition of the binary stream. The inferred composition of the binary stream may provide real-time or near real-time information to monitor the separation process to identify and locate any disruptions in the process.

In another embodiment, pressure-compensated temperatures may also be used at various locations in a distillation column to monitor and control the separation processes. For example, analyzers may be placed at various locations of the distillation columns, e.g., at various trays, to monitor the composition of the binary system. The inferred composition of the binary system may provide immediate information to the separation process and the parameters of the columns may be adjusted accordingly.

Similarly, other online probes, e.g., conductivity, near inferred, may also be placed at specific locations of the distillation columns to provide composition data.

In a continuous industrial process for producing ethanol, the temperature and pressure of the separation units and separated streams may be continuously monitored. Thus, the compositional ranges may be determined based on information that is already monitored and inferred using that information to determine the binary stream composition. In addition, a liquid online density sensor may be used to measure the density of particular binary streams. Advantageously, embodiments of the present invention may monitor streams without analyzers to measure one compound or each compound in the stream. Embodiments of the present invention may be used in conjunction with such analyzers to provide process control information to efficiently control the separation of ethanol from the ethanol mixture. The analyzer may include conductivity sensor, gas chromatography, infrared spectroscopy, total organic carbon (TOC) analyzer, etc.

The binary stream density may be measured by a Coriolis-type mass flow meter, densitometer, pressure transducer, thermocouple, or thermistor. When the binary stream is an acetic acid-water stream, there is a maximum density for acetic acid concentration. The density may be measure either above or below the maximum density as necessary. When the density exceeds the maximum density, an error condition may be signaled indicating a disruption in the binary stream composition. A binary stream of ethanol and water does not have a maximum density, but rather the density decreases as ethanol concentrations increase.

There may be multiple separation processes that use combinations of columns and/or membranes to remove the impurities from the ethanol. During the separation, the process is most likely to have at least one binary stream that may be used to infer the binary stream composition based on the temperature, pressure, and/or density. In one embodiment of the present invention, there is at least one binary stream selected from the group consisting of acetic acid and water, and ethanol and water. Preferably, there are at least two binary streams, one of which is an acetic acid and water binary stream and the other an ethanol and water stream. Although the binary stream may also include the ethanol product stream that contains water, such as an azeotropic amount of water, the ethanol and water binary stream may comprise an intermediate stream.

In a continuous process operating under normal conditions may maintain a stable composition of the binary stream. A compositional trend may be established after measuring a minimum time on stream, such as from 5 to 50 hours. The compositional trend will vary depending on the binary stream and location within the separation system and may be used to set a baseline. Once a compositional trend is established, an upset in the reactor or columns may be inferred by monitoring a pressured compensated temperature, or temperature compensated density. For example, any statistically significant change from a target composition may trigger a control in the column and/or reactor parameters. Monitoring in compositional trend may detect where the location of the problem and reduce or eliminate the production of any off-spec ethanol.

In embodiments of the present invention, when the inferred composition of the binary stream indicates unacceptable levels the binary components, the process may respond by controlling at least one column parameter. The column parameters include adjusting the reflux ratio, residue to feed ratio, the distillate to feed ratio, the column temperature, the column pressure, reboiler energy input and combinations thereof. In some embodiments, in addition to at least one column parameter, the process may also respond by controlling at least one reactor parameter. The reactor parameters include reactant feed rate, recycle rate, reactor temperature, reactor pressure, hydrogen to acid molar ratio, or combinations thereof.

The process may also have varying levels of response to different binary stream compositions. This may avoid large disruptions to plant operations that result in shutdown and start up costs as well as loss of ethanol product due to small changes in the binary stream composition. The type of response may be determined by the plant operators and adjusted as necessary. A total system shutdown may be necessary when a binary stream composition comprises essentially one of the two components, while a slight binary stream composition change may require adjusting the reflux ratio. Audible and/or visual alerts may signal changes in the binary compositions.

The process of the present invention may be used with any hydrogenation process for producing ethanol. The materials, catalysts, reaction conditions, and separation processes that may be used in the hydrogenation of acetic acid are described further below.

### Conductivity

When separating the crude ethanol product, an embodiment of the present invention separates acetic acid such that very low amounts of acetic acid carry over with the ethanol. To provide efficient separation, it is preferred to remove each organic compound, such as acetic acid, once from the crude ethanol product. Acetic acid generally is one of the highest boiling point components and it is preferred to remove acetic acid as a liquid. The removed acetic acid may be recycled to the reactor to produce additional ethanol. Preferably, less than 1000 wppm acetic acid carries over with ethanol, and more preferably less than 600 wppm acetic acid. In terms of ranges, the acetic acid carry over with ethanol may range from 10 to 1000 wppm, e.g., from 50 to 600 wppm, or from 100 to 200 wppm. When larger amounts of acetic acid carry over with the ethanol, the acetic acid may be esterified to increase the light organic concentrations and reduce yields of ethanol. In addition, acetic acid may lead to fouling of water effluent from the process. Also, increased acetic acid carry over may lead to an undesirable pH increase of the recovered ethanol.

In one embodiment, the present invention monitors acetic acid concentration in the condensed distillate based on a measured conductivity. The condensed distillate is from a column in which acetic acid is removed in the residue. Due to the dissociation of acetic acid in water, a conductivity cell can be used to detect low concentrations of hydronium and/or acetate ions that are generated by the dissociation of acetic acid. However, in the production of ethanol, the condensed distillate would not carry a binary mixture of acetic acid and water. Instead, the condensed distillate is a non-binary mixture and contains several organic solvents, such as ethanol, ethyl acetate, acetaldehyde, acetone and/or diethyl acetal, and these solvents may result in a reduced conductivity response. The reduced response requires a more sensitive conductivity monitor. The ability of a solvent to separate oppositely charged ions affects the dissociation of acetic acid and in turns affects the conductivity of the solution. Ethanol as a solvent has a lower dielectric constant than water and has a significant impact on the dissociation of acetic acid. The condensed distillate comprises at least both water and ethanol, and thus both relative concentrations of each compound may affect the dissociation of acetic acid. In most embodiments, condensed distillate has an amount of ethanol that exceeds the water amount on a weight basis, and thus the dielectric constant of ethanol may affect the dissociation of acetic acid to a greater extent. Based on the conductivity measurement, embodiments of the present invention are able to provide a real-time or near real-time data that provides information sufficient to control the column and/or reactor parameters.

A conductivity sensor may be used to determine acetic acid concentration content by measuring the electrical conductivity of the fluid. Suitable conductivity sensors may be capable of measuring at least from 0.1 to 10 micromhos/cm. In some embodiments, the sensor may be an inductive sensor and no electrically conductive material is in contact with the condensed distillate. The sensor may measure the conductivity of the condensed distillate in the overhead accumulator, or a stream withdrawn from the overhead accumulator. The withdrawn stream may be a stream that is separate from the recovered ethanol and the separate stream is dedicated for monitoring. After monitoring, the separate stream may be returned to the accumulator. For purposes of the present invention the withdrawn stream is an aliquot portion of the condensed distillate.

Although the conductivity of the condensed distillate may be measured at any temperature or pressure, it is preferable to measure the conductivity at a constant temperatures, such as an ambient temperature of less than 25° C., e.g., less than 20° C. or less than 18° C. Acid concentration typically increases in conductivity at a rate of 1.0%/° C. to 1.6%/° C. For example, the overhead accumulator may operate at a temperature of about 60° C. which would lead to a conductivity measurement that is 40% to 64% higher than conductivity measurements at 20° C. Thus, it may be preferable to measure the conductivity of a withdrawn stream that may be cooled to ambient temperatures. More preferably, a dedicated withdrawn stream that is an aliquot portion of the condensed distillate may be cooled prior to the conductivity sensor.

In embodiments of the present invention, when the conductivity of the condensed distillate is at a level that indicates unacceptable levels of acetic acid concentration, the process may respond by controlling at least one column parameter. An unacceptable level may vary depending on the ethanol application, but generally acetic acid concentrations greater than 1000 wppm are unacceptable. At an ambient temperature, conductivities from 3.5 to 4.5 micromhos/cm may indicate the maximum acceptable acetic acid concentration in the condensed distillate. The column parameters include adjusting the reflux ratio, residue to feed ratio, distillate to feed ratio, column temperature, column pressure, reboiler energy input and combinations thereof. In some embodiments, in addition to at least one column parameter, the process may also respond by controlling at least one reactor parameter. The reactor parameters include reactant feed rate, recycle rate, reactor temperature, reactor pressure, vapor separation, gas feed partial pressure or combinations thereof.

The process may also have varying levels of response to different conductivity measurements. This may avoid large disruptions to plant operations that result in shutdown and start up costs as well as loss of ethanol product due to smaller acetic acid concentration increases. The type of response may be determined by the plant operators and adjusted as necessary. For example, a total system shutdown may be necessary when the conductivity exceeds 8 micromhos/cm, while increasing the reflux ratio may be necessary when the conductivity reaches 3.5 micromhos/cm. Audible and/or visual alerts may signal higher conductivity measurements.

Although lower conductivity values, and thus low acetic acid concentration, are desirable, very low conductivity values may also present a problem for the industrial production of ethanol. Very low conductivity values may be less than 0.5 microohms/cm, e.g., less than 0.1 microohms/cm. A very low conductivity value may indicate that the column is operating inefficiently and refluxing a large portion of the distillate than otherwise necessary. In other situations, a very low conductivity may indicate that the column is operating to remove more ethanol in the residue with the acetic acid and thus resulting in a loss of ethanol product. These low conductivity values may also prompt a response to control a column and/or reactor parameter. Depending on the plant operation, it may not be necessary to respond to the very low conductivity values.

In some embodiments, it may be desirable to monitor the conductivity trends instead of absolute conductivity values. In a continuous process operating under normal conditions may maintain a stable conductivity trend that has minor variations. A conductivity trend may be established after measuring a minimum time on stream, such as from 5 to 50 hours, to set a baseline conductivity value. Once a conductivity trend is established, an upset in the reactor or columns may be detected by monitoring a conductivity value that is outside of the conductivity trend. For example, any change that exceeds 10% from a constant conductivity value may trigger a control in the column and/or reactor parameters. Monitoring a trend in conductivity may detect any increases or decreases in acetic acid concentrations in the condensed distillate.

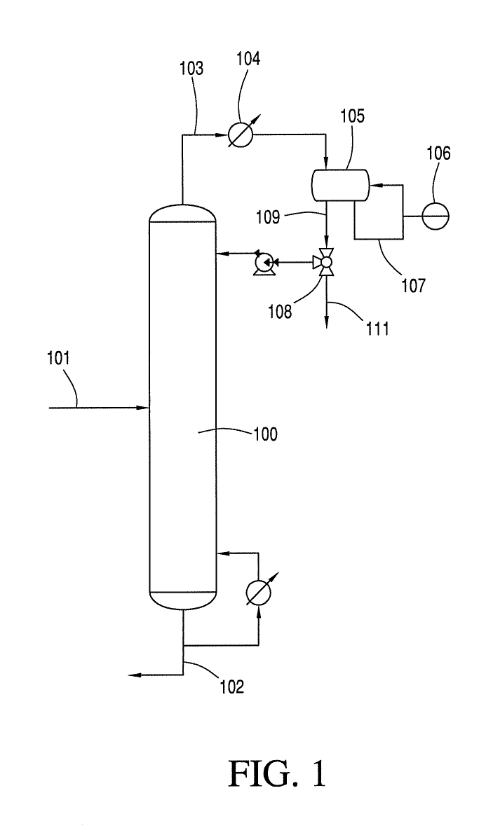

FIG. 1 shows a distillation column 100 in which a feed comprising ethanol, water, acetic acid, and ethyl acetate is fed via line 101. In one embodiment, the feed may be a crude ethanol product obtained from a hydrogenation reaction. The feed in line 101 may be obtained from hydrogenating acetic acid. Distillation column 100 operates to separate acetic acid in the residue which is withdrawn in line 102. Preferably, at least 95% of the acetic acid fed to the distillate column is withdrawn in the residue in line 102, and more preferably at least 99%. In some embodiments, a substantial portion of the water may also be withdrawn in the residue. The overhead vapors of column 100 in line 103 are condensed by passing through a condenser 104 and the condensed liquid is collected in overhead accumulator 105. As shown in FIG. 1, a conductivity sensor 106 is connected to an analysis stream 107 that is withdrawn from overhead accumulator 105.

Conductivity sensor 106 indirectly monitors the acetic acid concentration in the condensed distillate. The output of conductivity sensor 106 is partly connected to a control input of valve 108 and partly to a trigger input of an alarm unit (not shown). For example, the conductivity sensor 106 can be a device capable of measuring the conductivity of the condensed distillate. When above a predetermined conductivity value or when outside of a conductivity trend the process causes the valve 108 to close and hold up the liquid in accumulator 105 or to direct the withdrawn condensed distillate in line 109 to be refluxed via line 110. By closing the valve 108, the outlet is stopped and, as a result, the acetic acid does not carry over with the ethanol in line 111. Value may be opened to allow the condensed distillate in line 109 to flow through when the conductivity values are acceptable and ethanol may be further recovered from line 111 in one or more separation units. Preferably, ethanol is recovered with an acetic acid concentration that is less than 100 wppm, e.g., less than 50 wppm or less than 10 wppm. In addition, any effluent comprising water separated from line 111 also comprises low concentrations of acetic acid, e.g., of less than 600 wppm, e.g., less than 400 wppm or less than 200 wppm.

Although conductivity sensor 106 is shown as measuring stream 107, other conductivity sensors may be placed directly in distillation column 100 to measure the concentration of acetic acid at the specific locations.

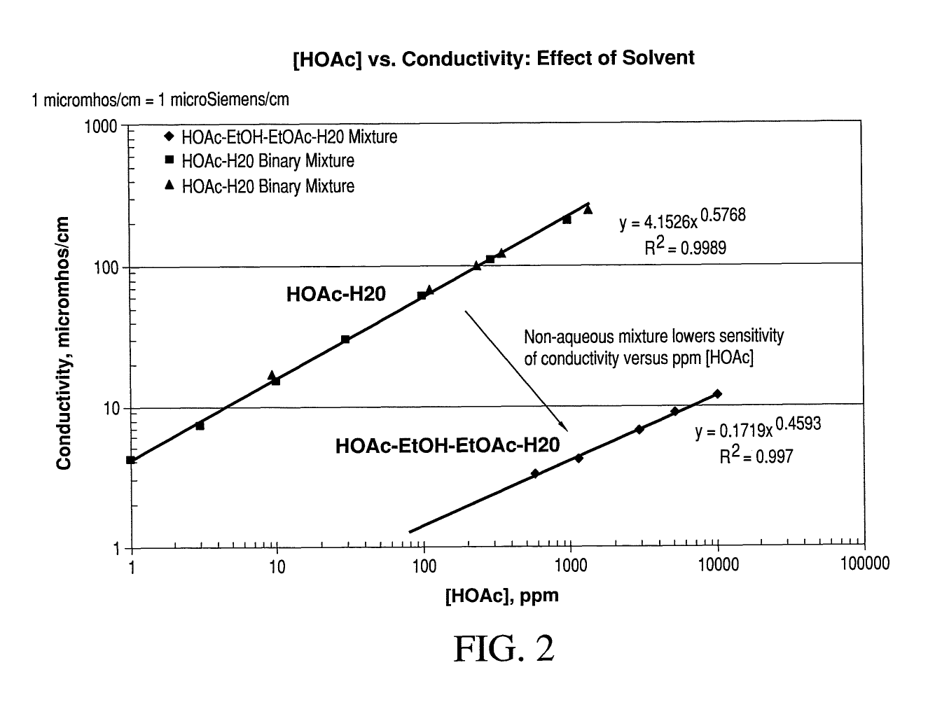

FIGS. 2-4 are graphs illustrating conductivity of streams. FIG. 2 is a graph of conductivity responses of a binary mixture and a non-aqueous mixture. The acetic acid/water binary mixtures are textbook data, which show that acetic acid concentration and conductivity has a linear relationship. The non-aqueous mixture shown on the graph includes ethanol, ethyl acetate, water, and acetic acid. The concentration of acetic acid in the non-aqueous mixture also has a linear relationship with conductivity. However, the non-aqueous mixture shows a lower sensitivity than the binary mixture of acetic acid/water.

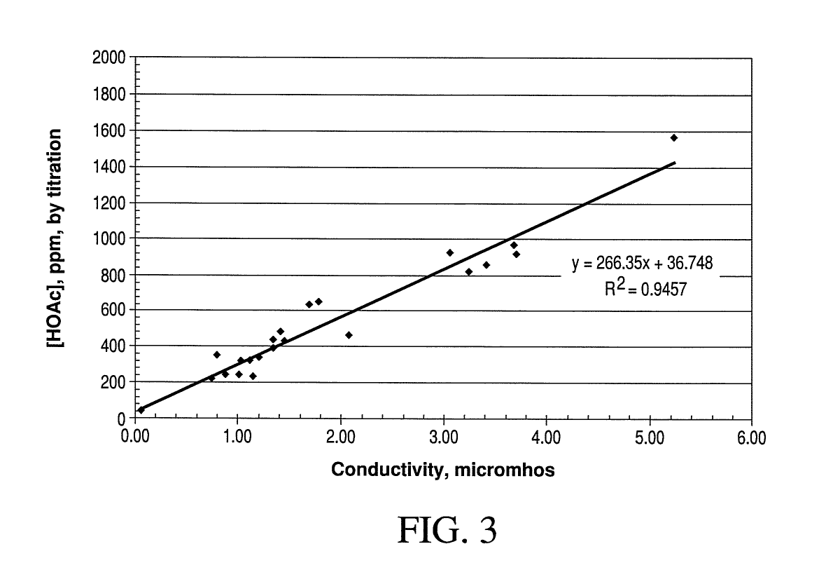

Samples from stream 107 were collected and conductivity of the samples were measured. The samples were also titrated to determine the concentration of the acetic acid in the sample. FIG. 3 is a graph of acetic acid concentration versus conductivity of stream 107 of FIG. 1. The linear regression suggests that the acetic acid concentration increases as conductivity increases. As such, by measuring the conductivity of the binary stream of the streams of the purification process, the concentration of acetic acid may be ascertained.

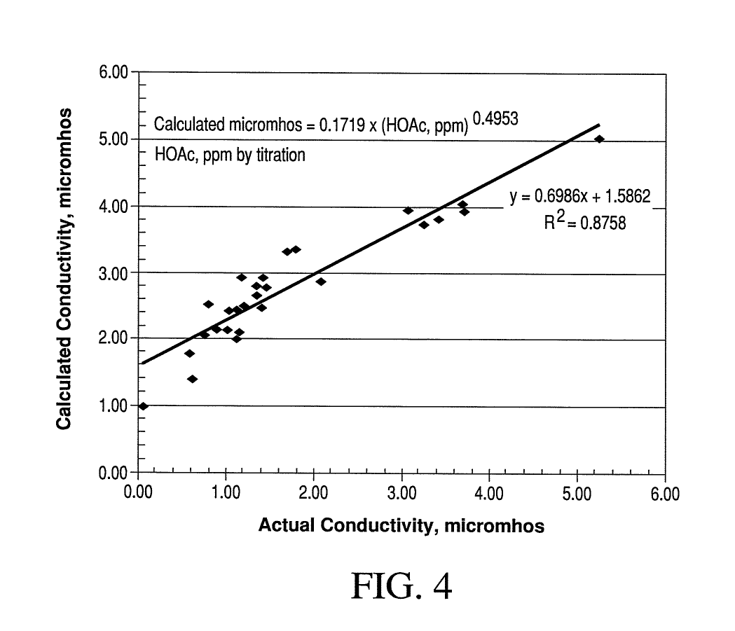

FIG. 4 is a graph comparing actual conductivity versus calculated conductivity of stream 107 of FIG. 1. The graph shows that the calculated conductivity provides a close representation of the actual conductivity of stream 107 of FIG. 1. By measuring the conductivity of the streams, the concentration of acetic acid in the stream may be calculated and adjustment to the purification may be made accordingly.

Other on-line analyzers may be used in place or in combination with the conductivity sensor 106. The process of the present invention may be used with any hydrogenation process for producing ethanol. The materials, catalysts, reaction conditions, and separation processes that may be used in the hydrogenation of acetic acid are described further below.

### Hydrogenation Conditions

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol stream may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Black liquor, which is an aqueous solution of lignin residues, hemicellulose, and inorganic chemicals, may also be used as a biomass source. Biomass-derived syngas has a detectable ¹⁴C isotope content as compared to fossil fuels such as coal or natural gas.

In another embodiment, the acetic acid used in the hydrogenation step may be formed from the fermentation of biomass. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as a syngas including hydrogen and carbon monoxide, are incorporated herein by reference in their entireties.

The acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol a variety of configuration using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa. The reactants may be fed to the reactor at a gas hourly space velocity (GHSV) ranging from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin silver/palladium, copper/palladium, copper/zinc, nickel/palladium, gold/palladium, ruthenium/rhenium, and ruthenium/iron. Exemplary catalysts are further described in U.S. Pat. No. 7,608,744 and U.S. Pub. No. 2010/0029995, the entireties of which are incorporated herein by reference. In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, Bi₂O₃.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group JIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain N or Pro. The Saint-Gobain N or Pro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197485 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least at least 40%, e.g., at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram catalyst per hour or from 600 to 2,000 grams of ethanol per kilogram catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol product produced by the hydrogenation process, before any subsequent processing, such as purification and separation, will typically comprise unreacted acetic acid, ethanol and water. As used herein, the term “crude ethanol product” refers to any composition comprising from 5 to 70 wt. % ethanol and from 5 to 40 wt. % water. In some exemplary embodiments, the crude ethanol product comprises ethanol in an amount from 5 to 70 wt. %, e.g., from 10 to 60 wt. %, or from 15 to 50 wt. %, based on the total weight of the crude ethanol product. Preferably, the crude ethanol product contains at least 10 wt. % ethanol, at least 15 wt. % ethanol or at least 20 wt. % ethanol. The crude ethanol product typically will further comprise unreacted acetic acid, depending on conversion, for example, in an amount of less than 90 wt. %, e.g., less than 80 wt. % or less than 70 wt. %. In terms of ranges, the unreacted acetic acid optionally is present in the crude ethanol product in an amount from 0 to 90 wt. %, e.g., from 5 to 80 wt. %, from 15 to 70 wt. %, from 20 to 70 wt. % or from 25 to 65 wt. %. As water is formed in the reaction process, water will generally be present in the crude ethanol product, for example, in amounts ranging from 5 to 40 wt. %, e.g., from 10 to 30 wt. % or from 10 to 26 wt. %.

Ethyl acetate may also be produced during the hydrogenation of acetic acid or through side reactions and may be present, for example, in amounts ranging from 0 to 30 wt. %, e.g., from 0 to 20 wt. %, from 1 to 12 wt. % or from 3 to 10 wt. %. Acetaldehyde may also be produced through side reactions and may be present, for example, in amounts ranging from 0 to 10 wt. %, e.g., from 0 to 3 wt. %, from 0.1 to 3 wt. % or from 0.2 to 2 wt. %. Other components, such as, for example, esters, ethers, aldehydes, ketones, alkanes, and carbon dioxide, if detectable, collectively may be present in amounts less than 10 wt. %, e.g., less than 6 wt. % or less than 4 wt. %. In terms of ranges, other components may be present in an amount from 0.1 to 10 wt. %, e.g., from 0.1 to 6 wt. %, or from 0.1 to 4 wt. %. Exemplary component ranges for the crude ethanol product are provided in Table 1.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 70 | 15 to 70 | 15 to 50 | 25 to 50 |
| Acetic Acid | 0 to 90 | 0 to 80 | 15 to 70 | 20 to 70 |
| Water | 5 to 40 | 5 to 30 | 10 to 30 | 10 to 26 |
| Ethyl Acetate | 0 to 30 | 0 to 20 | 1 to 18 | 3 to 12 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product comprises acetic acid in an amount less than 20 wt. %, e.g., less than 15 wt. %, less than 10 wt. % or less than 5 wt. %. In embodiments having lower amounts of acetic acid, the conversion of acetic acid is preferably greater than 50%, e.g., greater than 75% or greater than 90%. In addition, the selectivity to ethanol may also be preferably high, and is preferably greater than 50%, e.g., greater than 75% or greater than 90%.

### Purification

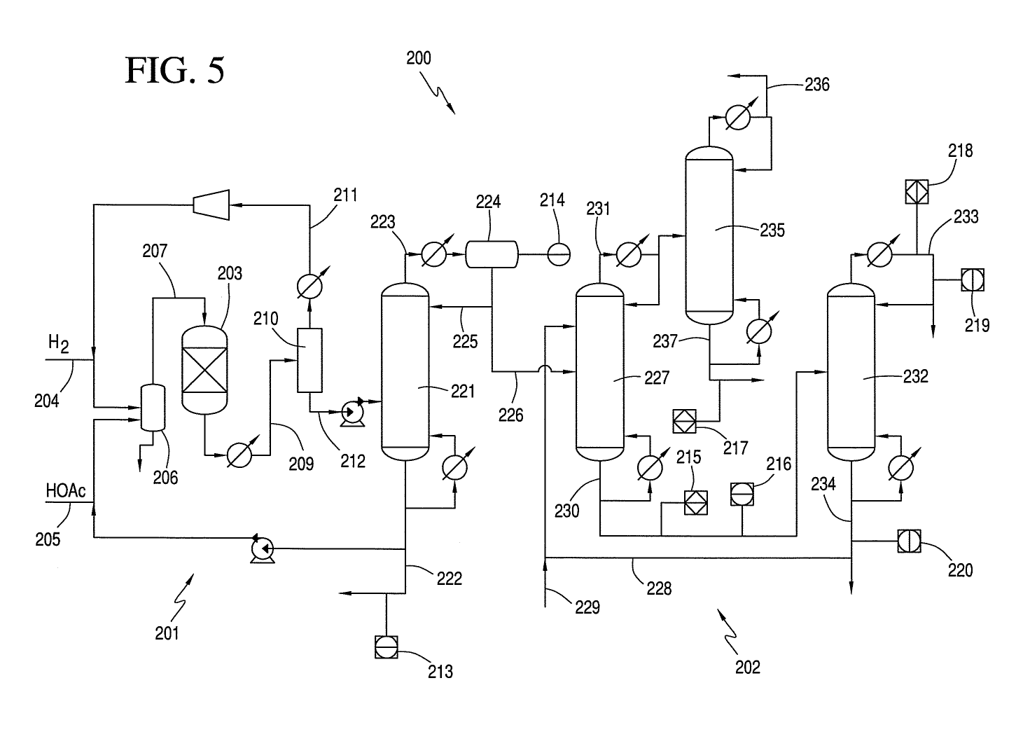

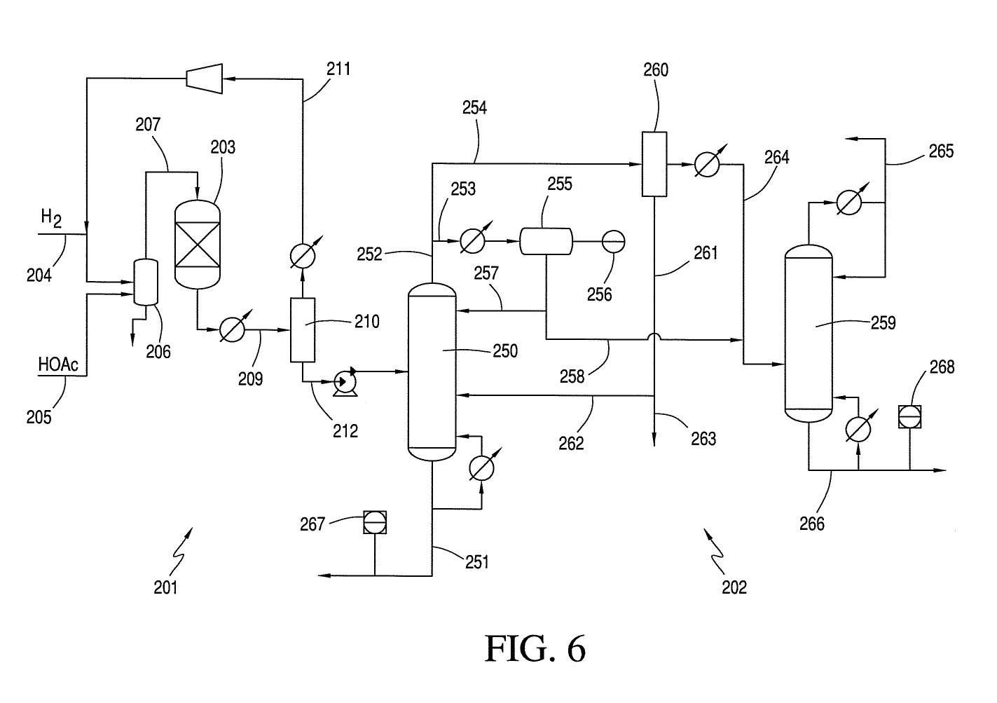

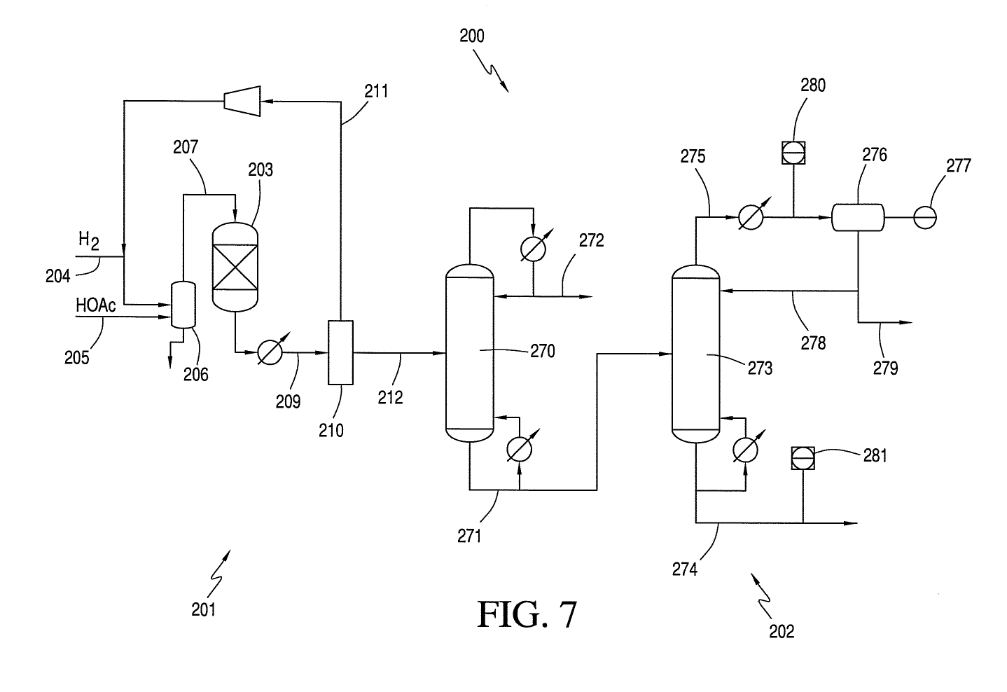

Ethanol produced may be recovered using several different techniques. In general, the hydrogenation system 200 comprises reaction zone 201 and distillation zone 202. The distillation zone 202 may comprise at least one column as shown in FIG. 5. In FIG. 5, six streams that may be monitored to control and regulate the hydrogenation and separation process. For example, sensors 213, 216, 219, and 220 may be used to monitor the binary streams; on-line analyzers 215, 217, and 218 may be used to monitor light ends column 227 to prevent the buildup of impurities in the ethanol product. Specifically, the on-line analyzers 215, 217, and 218 may be used to control the column parameter of light ends column 227, and the acetaldehyde removal column 235 to prevent the buildup of impurities in the impurities with the ethanol product and/or ethyl acetate co-product. In FIG. 6, the crude ethanol mixture is separated in two columns with an intervening water separation. In FIG. 7, the separation of the crude ethanol mixture uses two columns and the conductivity is monitored after the second column. In addition, in FIGS. 6 and 7, there are two binary streams that may be monitored to control and regulate the hydrogenation and separation process. Although all of these binary streams may be monitored, in some embodiments, at least one of the binary streams may be monitored to provide real-time or near real-time process control information. The concentrations of the components of the binary streams may be inferred, without direct measurement, by utilizing the known density, temperature, and pressure of the binary streams. In other embodiment, other on-line analyzer For purposes of convenience, the columns in each exemplary separation processes, may be referred as the first column, second columns, third columns, etc.

The on-line analyzers shown in FIGS. 5, 6, and 7 may be any suitable analyzer as described above that is capable of measuring the concentration of at least one organic impurity. The on-line analyzer may be used to measure at least one organic impurity in any stream in the purification process. Preferably, the on-line analyzer is capable of detecting very low concentrations in the stream, e.g., less than 100 wppm, e.g., less than 10 wppm, less than 1 wppm or less than 0.1 wppm. Although the on-line sensor may measure the concentration of ethanol, it is more preferred to measure the concentration of the organic impurities to control the process. In another embodiment, on-line analyzes may also be placed directly in the distillation columns, for example, at specific trays, to monitor the purification process. As such, adjustments to the purification condition may be modified.

Density generally decreases with increasing temperature and increases with decreasing temperature. By knowing the density and temperature of a binary stream, its composition may be inferred. Table 2 below is an example of a series of data depicting the concentration (in wt. %) of ethanol vs. the density (g/cm³) at 20° C. and 35° C. Thus, by measuring the density of the mixture at a known temperature, the concentration of the binary stream may be determined.

TABLE 2
Temperature Effect on Density of Binary Ethanol-Water Mixtures

| Concentration of | Density at 20° C. | Density at 35° C. |
|---|---|---|
| Ethanol (wt. %) | (g/cm³) | (g/cm³) |
| 0 | 0.99823 | 0.99406 |
| 10 | 0.98187 | 0.97685 |
| 20 | 0.96864 | 0.96134 |
| 30 | 0.95382 | 0.94403 |
| 40 | 0.93518 | 0.92385 |
| 50 | 0.91384 | 0.90168 |
| 60 | 0.89113 | 0.87851 |
| 70 | 0.86766 | 0.85470 |
| 80 | 0.84344 | 0.83029 |
| 90 | 0.81797 | 0.80478 |
| 100 | 0.78934 | 0.77641 |

Table 3 is an example of a series of data depicting the concentration of acetic acid (in wt. %) vs. density (g/ml) at 110° C. and 140° C. According to Table 3, the density of the binary mixture decreases as the concentration of acetic acid increases until the binary mixture reaches maximum density. As shown in Table 3, the maximum density of the binary mixture is between 60 wt. % and 70 wt. %.

TABLE 3
Temperature Effect on Density of Binary
Acetic Acid - Water Mixtures

| Concentration of | Density at 110° C. | Density at 140° C. |
|---|---|---|
| Acetic Acid (wt. %) | (g/ml) | (g/ml) |
| 0 | 0.9500 | 0.9245 |
| 10 | 0.9563 | 0.9317 |
| 20 | 0.9620 | 0.9364 |
| 30 | 0.9684 | 0.9420 |
| 40 | 0.9749 | 0.9472 |
| 50 | 0.9795 | 0.9500 |
| 60 | 0.9820 | 0.9521 |
| 70 | 0.9820 | 0.9521 |
| 80 | 0.9762 | 0.9442 |
| 90 | 0.9642 | 0.9300 |
| 100 | 0.9482 | 0.9124 |

In each of the figures, hydrogen and acetic acid via lines 204 and 205, respectively, are fed to a vaporizer 206 to create a vapor feed stream in line 207 that is directed to reactor 203. In one embodiment, lines 204 and 205 may be combined and jointly fed to the vaporizer 206. The temperature of the vapor feed stream in line 207 is preferably from 100° C. to 350° C., e.g., from 120° C. to 310° C. or from 150° C. to 300° C. Any feed that is not vaporized is removed from vaporizer 206 and may be recycled or discarded. In addition, although line 207 is shown as being directed to the top of reactor 203, line 207 may be directed to the side, upper portion, or bottom of reactor 203.

Reactor 203 contains the catalyst that is used in the hydrogenation of the carboxylic acid, preferably acetic acid. In one embodiment, one or more guard beds (not shown) may be used upstream of the reactor, optionally upstream of vaporizer 206, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a crude ethanol mixture stream is withdrawn, preferably continuously, from reactor 203 via line 209.

The crude ethanol mixture stream in line 209 may be condensed and fed to a separator 210, which, in turn, provides a vapor stream 211 and a liquid stream 212. In some embodiments, separator 210 may comprise a flasher or a knockout pot. The separator 210 may operate at a temperature of from 20° C. to 250° C., e.g., from 30° C. to 225° C. The pressure of separator 210 may be from 50 kPa to 2000 kPa, e.g., from 75 kPa to 1500 kPa. Optionally, the crude ethanol mixture in line 209 may pass through one or more membranes to separate hydrogen and/or other non-condensable gases.

The vapor stream 211 exiting separator 210 may comprise hydrogen and hydrocarbons, and may be purged and/or returned to reaction zone 201. When returned to reaction zone 201, vapor stream 210 is combined with the hydrogen feed 204 and co-fed to vaporizer 206. In some embodiments, the returned vapor stream 211 may be compressed before being combined with hydrogen feed 204.

In FIG. 5, the liquid from separator 210 is withdrawn and pumped as a feed composition via line 212 to the side of first column 221, also referred to as the “acid separation column.” The contents of line 212 typically will be substantially similar to the product obtained directly from the reactor, and may, in fact, also be characterized as a crude ethanol product. However, the feed composition in line 212 preferably has substantially no hydrogen, carbon dioxide, methane or ethane, which are removed by separator 210. Exemplary components of liquid in line 212 are provided in Table 4. It should be understood that liquid line 212 may contain other components, not listed, such as components in the feed.

TABLE 4
FEED COMPOSITION

| | Conc. (wt. %) | Conc. (wt. %) | Conc. (wt. %) |
|---|---|---|---|
| Ethanol | 5 to 70 | 10 to 60 | 15 to 50 |
| Acetic Acid | <90 | 5 to 80 | 15 to 70 |
| Water | 5 to 35 | 5 to 30 | 10 to 30 |
| Ethyl Acetate | <20 | 0.001 to 15 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout the present specification may not be present and if present may be present in amounts greater than 0.0001 wt. %.

The “other esters” in Table 4 may include, but are not limited to, ethyl propionate, methyl acetate, isopropyl acetate, n-propyl acetate, n-butyl acetate or mixtures thereof. The “other ethers” in Table 4 may include, but are not limited to, diethyl ether, methyl ethyl ether, isobutyl ethyl ether or mixtures thereof. The “other alcohols” in Table 4 may include, but are not limited to, methanol, isopropanol, n-propanol, n-butanol or mixtures thereof. In one embodiment, the feed composition, e.g., line 212, may comprise propanol, e.g., isopropanol and/or n-propanol, in an amount from 0.001 to 0.1 wt. %, from 0.001 to 0.05 wt. % or from 0.001 to 0.03 wt. %. In should be understood that these other components may be carried through in any of the distillate or residue streams described herein and will not be further described herein, unless indicated otherwise.

Optionally, crude ethanol mixture in line 209 or in liquid stream 212 may be further fed to an esterification reactor, hydrogenolysis reactor, or combination thereof. An esterification reactor may be used to consume residual acetic acid present in the crude ethanol mixture to further reduce the amount of acetic acid that would otherwise need to be removed. Hydrogenolysis may be used to convert ethyl acetate in the crude ethanol mixture to ethanol.

In the embodiment shown in FIG. 5, line 212 is introduced in the lower part of first column 221, e.g., lower half or lower third. When first column 221 is operated under standard atmospheric pressure, the temperature of the residue exiting in line 222 preferably is from 95° C. to 120° C., e.g., from 105° C. to 117° C. or from 110° C. to 115° C. The temperature of the distillate exiting in line 223 preferably is from 70° C. to 110° C., e.g., from 75° C. to 95° C. or from 80° C. to 90° C. In other embodiments, the pressure of first column 221 may range from 0.1 kPa to 510 kPa, e.g., from 1 kPa to 475 kPa or from 1 kPa to 375 kPa. In first column 221, a binary stream comprising acetic acid and water is withdrawn, preferably continuously, as a residue in line 222. The binary stream in line 222 should contain only minor amounts of ethanol and other impurities. One or more sensors 213 may measure the temperature, pressure, and/or density of residue in line 222. The composition of the binary stream in line 222 may be determined by measuring a temperature-compensated density and/or by measuring a pressure-compensated temperature. In one embodiment, the one or more sensors 213 may measure at least the temperature of binary stream in line 222 and either the density or pressure.

The binary stream in line 222 comprises at least 60 wt. % acetic acid with the balance being water. The composition may vary depending on the reactor conditions, catalyst, and selectivity. In one embodiment, binary stream in line 222 comprises from 85 to 95 wt. % acetic acid and 5 to 15 wt. % water. When the system detects a binary composition that indicates less acetic acid than expected, it may indicate that acetic acid is being carried over with the ethanol. The binary stream composition may be used to increase the reflux at the top of column 221 or purge the overhead stream as an off-spec product. Lower than expected concentrations of acetic acid in the binary stream may also indicate the presence of ethanol and/or water in the residue. This also is undesirable and the process may increase the reboiler temperature so that the ethanol is removed in the overhead. For purposes of clarity valves, controllers, regulators, etc., that may be used to adjust the column and/or reactor parameters are not shown in FIG. 5.

At temperatures between 110° C. and 140° C., the maximum density of acetic acid and water is around 70 wt. % acetic acid. The maximum density may be less than 0.98 g/ml, e.g., less than 0.96 g/ml, or less than 0.95 g/ml. Thus, acetic acid concentration is determined by using the temperature-compensated density measurements above the maximum density.

In one embodiment, the pressure-compensated temperature may allow a temperature-rate controller to be used in the base or stripping section of column 221. The temperatures in the base of column 221 have a correlation to the water concentration in the binary stream withdrawn in line 222.

The compositions of the binary stream in line 222 may determine where the stream is directed. The following are exemplary processes for further treating the residue in line 222 and it should be understood that any of the following may be used regardless of acetic acid concentration. When the residue comprises a majority of acetic acid, e.g., greater than 70 wt. %, the residue may be recycled to the reactor without any separation of the water. In one embodiment, the residue may be separated into an acetic acid stream and a water stream when the residue comprises a majority of acetic acid, e.g., greater than 50 wt. %. Acetic acid may also be recovered in some embodiments from the residue having a lower acetic acid concentration. The residue may be separated into the acetic acid and water streams by a distillation column or one or more membranes. If a membrane or an array of membranes is employed to separate the acetic acid from the water, the membrane or array of membranes may be selected from any suitable acid resistant membrane that is capable of removing a permeate water stream. The resulting acetic acid stream optionally is returned to the hydrogenation reactor. The resulting water stream may be used as an extractive agent or to hydrolyze an ester-containing stream in a hydrolysis unit.

Some or all of the residue may be returned and/or recycled back to reaction zone 201 via line 222. Recycling the acetic acid in line 222 to vaporizer 206 may reduce the amount of heavies that need to be purged from vaporizer 206. Reducing the amount of heavies to be purged may improve efficiencies of the process while reducing byproducts.

First column 220 also forms an overhead distillate, which is withdrawn in line 223, is condensed and collected in an overhead accumulator 224. As shown in FIG. 5, a conductivity sensor 214 directly monitors the conductivity of the condensed distillate in accumulator 224. The condensed distillate in accumulator 224 comprises primarily ethanol, as well as water, ethyl acetate, acetaldehyde, and/or diethyl acetal. In other embodiments, conductivity sensor 214 may measure a withdrawn stream from accumulator 224 as described above in FIG. 1. As discussed above, depending on the conductivity measurement, one or more column and/or reactor parameters may be adjust such that the acetic acid levels are acceptable in the condensed distillate. For purposes of clarity valves, controllers, regulators, etc., that may be used to adjust the column and/or reactor parameters are not shown in FIGS. 5-7. A portion of the condensed distillate may be refluxed via line 225 at a ratio of from 10:1 to 1:10, e.g., from 3:1 to 1:3 or from 1:2 to 2:1. Acetic acid concentration in the first distillate preferably is from 10 to 1000 wppm, e.g., from 50 to 600 wppm, or from 100 to 200 wppm. The remaining portion of the condensed distillate is withdrawn via line 226 and directed to a second column 227.

Exemplary components of the distillate and residue compositions for first column 221 are provided in Table 5 below. It should also be understood that the distillate and residue may also contain other components, not listed, such as components in the feed. For convenience, the distillate and residue of the first column may also be referred to as the “first distillate” or “first residue.” The distillates or residues of the other columns may also be referred to with similar numeric modifiers (second, third, etc.) to distinguish them from one another, but such modifiers should not be construed as requiring any particular separation order.

TABLE 5
FIRST COLUMN 221 (FIG. 5)

| | Conc. (wt. %) | Conc. (wt. %) | Conc. (wt. %) |
|---|---|---|---|
| Distillate | | | |
| Ethanol | 20 to 75 | 30 to 70 | 40 to 65 |
| Water | 10 to 40 | 15 to 35 | 20 to 35 |
| Ethyl Acetate | <60 | 5.0 to 40 | 10 to 30 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | 60 to 100 | 70 to 95 | 85 to 92 |
| Water | <30 | 1 to 20 | 1 to 15 |
| Ethanol | <1 | <0.9 | <0.07 |

Acetic acid concentration in the distillate may vary and conductivity sensor 214 detects when the acetic acid concentration is outside of acceptable levels. Acceptable levels of acetic acid concentration may range from 10 to 1000 wppm, e.g., from 50 to 600 wppm, or from 100 to 200 wppm. More or less acetic acid may be present in the first distillate in line 223 and will be detected by conductivity sensor 214 prior to forwarding the condensed distillate to second column 227. One or more column and/or reactor parameters may be adjusted such that the acetic acid concentration levels return to acceptable levels. Conductivity sensor 214 provides monitoring to allow for ethanol recovery with acceptable levels of acetic acid.

To further recover ethanol, condensed distillate in line 226 is introduced to the second column 227, also referred to as the “light ends column,” preferably in the middle part of column 227, e.g., middle half or middle third. As one example, when a 25 tray column is utilized in a column without water extraction, line 226 is introduced at tray 17. Light ends column 227 removes the light organics in the second distillate 231 to recover a diluted ethanol stream in the residue stream 230. On-line analyzer 215 may measure the concentration of at least one impurity to control the column parameters of light ends column 227.

The residue stream 230 comprises mainly ethanol and water. On-line analyzer 215 may be used to determine the amount of impurities in the residue stream 230 and to control the separation parameter of the light ends column 227. As shown in Table 6, different compounds have different signature absorption frequency values based on their structures. As such, an infrared on-line analyzer using fiber optics may be programmed to measure absorption values over a range of frequencies that enables calculation of specific organic compound concentrations contained in the mixture. The results may be used to augment the operating parameters of the light ends column 227. For example, if the on-line analyzer detects ethyl acetate or acetaldehyde in an amount greater than a predetermined amount, it may indicate that a higher reflux ratio or higher temperature is needed for the distillation column. And the system may be adjusted accordingly. Similarly, if the amount of ethyl acetate or acetaldehyde is detected in the residue stream 230 is too low, it may suggest that a larger than desired amount of ethanol is being refluxed. Therefore, the amount of ethanol product collected downstream is reduced and the system may need to be adjusted to optimize the recovery of ethanol.

TABLE 6

| Compound | Stretch and Frequency |
|---|---|
| Ethanol | O—H stretch, from 3500 to 3200 cm⁻¹ |
| | C—O stretch, from 1260 to 1050 cm⁻¹ |
| Ethyl Acetate | C═O stretch, from 1750 to 1735 cm⁻¹ |
| | C—O stretch, from 1300 to 1000 cm⁻¹ |
| Acetaldehyde | H—C═O stretch, from 2830 to 2695 cm⁻¹ |
| | C═O stretch, from 1740 to 1720 cm⁻¹ |
| Acetal | C—O stretch, from 1600 to 1000 cm⁻¹ |
| Acetone | C═O stretch, from 1820 to 1670 cm⁻¹ |

In an embodiment, gas chromatograph may also be used as on-line analyzer 215 to detect the amount of components in second residue 230. Table 7 shows the detection ranges of the on-line analyzers for different impurities. The concentrations of one or more impurities may be used to determine whether the column parameter for second column 227 requires any augmentation.

TABLE 7

| Impurities | Detection Ranges |
|---|---|
| Ethyl acetate | 10-100 wppm |
| Acetaldehyde | 1-20 wppm |
| Diethyl acetate | 1-20 wppm |
| n-propanol | 5-100 wppm |
| Isopropanol | 5-100 wppm |
| n-butanol | 1-20 wppm |

In one embodiment, preferably the second column 227 is an extractive distillation column, and an extraction agent is added thereto via lines 228 and/or 229. Extractive distillation is a method of separating close boiling components, such as azeotropes, by distilling the feed in the presence of an extraction agent. The extraction agent preferably has a boiling point that is higher than the compounds being separated in the feed. In preferred embodiments, the extraction agent is comprised primarily of water. As indicated above, the condensed distillate in line 226 that is fed to the second column 227, which comprises ethyl acetate, ethanol, and water. These compounds tend to form binary and ternary azeotropes, which decrease separation efficiency. As shown, in one embodiment the extraction agent comprises the third residue in line 228. Preferably, the recycled third residue in line 228 is fed to second column 227 at a point higher than the condensed distillate in line 226. In one embodiment, the recycled third residue in line 228 is fed near the top of second column 227 or fed, for example, above the feed in line 226 and below the reflux line from the condensed overheads. In a tray column, the third residue in line 228 is continuously added near the top of the second column 227 so that an appreciable amount of the third residue is present in the liquid phase on all of the trays below. In another embodiment, the extraction agent is fed from a source outside of the process 200 via line 229 to second column 227. Preferably this extraction agent comprises water.

The molar ratio of the water in the extraction agent to the ethanol in the feed to the second column is preferably at least 0.5:1, e.g., at least 1:1 or at least 3:1. In terms of ranges, preferred molar ratios may range from 0.5:1 to 8:1, e.g., from 1:1 to 7:1 or from 2:1 to 6.5:1. Higher molar ratios may be used but with diminishing returns in terms of the additional ethyl acetate in the second distillate and decreased ethanol concentrations in the second column distillate.

In one embodiment, an additional extraction agent, such as water from an external source, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N-dimethyl-1,3-propanediamine, N,N′-dimethylethylenediamine, diethylene triamine, hexamethylene diamine and 1,3-diaminopentane, an alkylated thiopene, dodecane, tridecane, tetradecane and chlorinated paraffins, may be added to second column 227. Some suitable extraction agents include those described in U.S. Pat. Nos. 4,379,028, 4,569,726, 5,993,610 and 6,375,807, the entire contents and disclosure of which are hereby incorporated by reference. The additional extraction agent may be combined with the recycled third residue in line 228 and co-fed to the second column 227. The additional extraction agent may also be added separately to the second column 227. In one aspect, the extraction agent comprises an extraction agent, e.g., water, derived from an external source via line 229 and none of the extraction agent is derived from the third residue.

In one optional embodiment, there may be a conductivity sensor for monitoring the acetic acid concentration in the second residue in line 230.

Second column 227 may be a tray or packed column. In one embodiment, second column 227 is a tray column having from 5 to 70 trays, e.g., from 15 to 50 trays or from 20 to 45 trays. Although the temperature and pressure of second column 227 may vary, when at atmospheric pressure the temperature of the second residue exiting in line 230 preferably is from 60° C. to 90° C., e.g., from 70° C. to 90° C. The temperature of the second distillate exiting in line 231 from second column 227 preferably is from 50° C. to 90° C., e.g., from 60° C. to 80° C. Column 227 may operate at atmospheric pressure. In other embodiments, the pressure of second column 227 may range from 0.1 kPa to 510 kPa.

In second column 227, when water is used as an extractive agent, a binary stream comprising ethanol and water is withdrawn, preferably continuously, as a residue in line 230. The binary stream in line 230 should contain only minor amounts of ethyl acetate and other impurities. One or more sensors 216 may measure the temperature, pressure, and/or density of residue in line 230. The composition of the binary stream in line 230 may be determined by measuring a temperature-compensated density and/or by measuring a pressure-compensated temperature. In one embodiment, the one or more sensors 216 may measure at least the temperature of binary stream in line 230 and either the density or pressure.

Ethanol and water in a binary stream have a large density difference that allows for accurate and real-time compositional information of the binary stream. A liquid density measurement is sensitive to temperature changes and thus the temperature of the binary stream in line 230 may also be determined.

Vapor-liquid equilibrium data may also be used to infer the composition of an ethanol-water binary stream based on the pressure-compensated temperate. The compositional changes are most responsive to lower ethanol concentrations and less responsive to higher ethanol concentrations. When an extractive agent is used, primarily water, the concentration of ethanol in line 230 may be expected to be between 20 wt. % to 50 wt. %, e.g., 25 wt. % to 35 wt. %. Thus, the pressure-compensated temperature inferred composition may be used to determine the composition of the binary stream in line 230.

When sensors 216 determine a composition of the binary stream is outside of the trend or expected value, the system 200 may regulate second column 227. For example, when ethanol concentrations are higher than expected, additional water as an extractive agent may be fed to second column 227. Likewise, lower ethanol concentration may indicate that too much extractive agent is being fed and the system may reduce the flow the extractive agent. In some embodiments, sensors 216 may be used in combination with sensors 213 for the first column. Using the water concentration based on the data from sensors 213 may be used to set an expected water concentration, or baseline value, to be determined by sensors 216.

Exemplary components for the distillate and residue compositions for second column 227 are provided in Table 8 below. In some embodiments, the second distillate in line 231 may be a non-binary stream. It should be understood that the distillate and residue may also contain other components, not listed, such as components in the feed.

TABLE 8
SECOND COLUMN 227 (FIG. 5)

| | Conc. (wt. %) | Conc. (wt. %) | Conc. (wt. %) |
|---|---|---|---|
| Distillate | | | |
| Ethyl Acetate | 10 to 99 | 25 to 95 | 50 to 93 |
| Acetaldehyde | <25 | 0.5 to 15 | 1 to 8 |
| Water | <25 | 0.5 to 20 | 4 to 16 |
| Ethanol | <30 | 0.001 to 15 | 0.01 to 5 |
| Acetal | <5 | 0.001 to 2 | 0.01 to 1 |
| Residue | | | |
| Water | 30 to 90 | 40 to 85 | 50 to 85 |
| Ethanol | 10 to 75 | 15 to 60 | 20 to 50 |
| Ethyl Acetate | <3 | 0.001 to 2 | 0.001 to 0.5 |
| Acetic Acid | <0.5 | 0.001 to 0.3 | 0.001 to 0.2 |
| Diethyl Acetal | <0.1 | <0.025 | <0.002 |
| Acetaldehyde | <0.1 | <0.025 | <0.002 |
| Diethyl ether | <0.1 | <0.025 | <0.002 |
| n-propanol | <0.1 | <0.05 | <0.01 |
| Isopropanol | <0.1 | <0.05 | <0.01 |
| n-butanol | <0.1 | <0.025 | <0.002 |

In one embodiment, the second residue in line 230 preferably comprises substantially no acetic acid. The second residue in line 230 may comprise less than 0.1 wt. % ethyl acetate, less than 0.05 wt. %, or less than 0.01 wt. %.

Although increased concentrations of impurities may be present in the residue in line 230, it is preferred to maintain these impurities below the acceptable baseline levels. One or more sensors 215 may measure the impurity concentration and adjust the column parameters accordingly to reduce the impurity concentration. In addition, measuring the impurity concentration may prevent production of off-spec product by shutting down the reactor and/or column when large amounts of impurities build up in the second residue in line 230. The off-spec residue in line 230 may be also be discarded or sent to a recycle vessel for subsequent processing.

As shown, the second residue from second column 227, which comprises ethanol and water, is fed via line 230 to third column 232, also referred to as the “product column.” More preferably, the second residue in line 230 is introduced in the lower part of third column 232, e.g., lower half or lower third. Third column 232 recovers ethanol, which preferably is substantially pure with respect to organic impurities and other than the azeotropic water content, as the distillate in line 233. The distillate of third column 233 preferably is refluxed as shown in FIG. 5, for example, at a reflux ratio of from 1:10 to 10:1, e.g., from 1:3 to 3:1 or from 1:2 to 2:1. The third distillate in line 233 may contain approximately an azeotropic amount of water, e.g., less than 12 wt. %. To further monitor the process, one or more on-line analyzers 218 or 219 may measure the impurity concentration in the third distillate in line 233. The impurity concentration may also be used to adjust at least one column parameter of the light ends column 227. The impurity concentration in the third distillate is expected to be greater due to the concentration of ethanol in third column 232.

Similar to on-line analyzer 215, on-line analyzer 218 may be an infrared spectrometer, a gas chromatograph, or other analyzers that is capable of measuring the concentration of at least one impurities in the third distillate in line 233. Table 9 shows the detection ranges of on-line analyzers for different impurities in the third distillate in line 233.

TABLE 9

| Compound | Detection Ranges |
|---|---|
| Ethyl acetate | 10-100 wppm |
| Acetaldehyde | 1-100 wppm |
| Diethyl acetate | 10-200 wppm |
| n-propanol | 10-100 wppm |
| Isopropanol | 10-200 wppm |
| n-butanol | 1-20 wppm |

In preferred embodiments, the recycling of the third residue in line 234 promotes the separation of ethyl acetate from the residue of second column 227. For example, the weight ratio of ethyl acetate in the second residue to second distillate preferably is less than 0.4:1, e.g., less than 0.2:1 or less than 0.1:1. In embodiments that use an extractive distillation column with water as an extraction agent as second column 227, the weight ratio of ethyl acetate in the second residue to ethyl acetate in the second distillate approaches zero.

The weight ratio of ethanol in the second residue to second distillate preferably is at least 3:1, e.g., at least 6:1, at least 8:1, at least 10:1 or at least 15:1. All or a portion of the third residue is recycled to the second column. In one embodiment, all of the third residue may be recycled until process 200 reaches a steady state and then a portion of the third residue is recycled with the remaining portion being purged from the system 200. The composition of the second residue will tend to have lower amounts of ethanol than when the third residue is not recycled. As the third residue is recycled, the composition of the second residue, as provided in Table 8, comprises less than 30 wt. % of ethanol, e.g., less than 20 wt. % or less than 15 wt. %. The majority of the second residue preferably comprises water. Notwithstanding this effect, the extractive distillation step advantageously also reduces the amount of ethyl acetate that is sent to the third column, which is highly beneficial in ultimately forming a highly pure ethanol product.

In one embodiment, the distillate in line 233 may be a binary stream and the composition of the binary stream may be measured based on data obtained using one or more sensors 219 to measure the temperature, pressure, and/or density of the stream. The distillate in line 233 may contain approximately an azeotropic amount of water, e.g., less than 12 wt. %. A temperature-compensated density measurement may be preferred to measure the expected higher ethanol concentrations.

Based on the water concentration in line 233, the system 200 may determine if it is necessary to further dehydrate the recovered ethanol to produce a suitable ethanol product. The third distillate in line 233 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit.

The third residue in line 234, which comprises primarily water, preferably is returned to the second column 227 as an extraction agent as described above. Although the third residue in line 234 is not a binary stream because low concentrations of ethanol and/or acetic acid, the density of third residue in line 234 may be measured using one or more sensors 220. Preferably, these sensors measure the density in line 234 to monitor whether the third residue being recycled contains water.

Although FIG. 5 shows third residue being directly recycled to second column 227, third residue may also be returned indirectly, for example, by storing a portion or all of the third residue in a tank (not shown) or treating the third residue to further separate any minor components such as aldehydes, higher molecular weight alcohols, or esters in one or more additional columns (not shown).

The third residue in line 234, which comprises primarily water, preferably is returned to the second column 227 as an extraction agent as described above. In another embodiment, a portion of the third residue may be used to hydrolyze any other stream, such as one or more streams comprising ethyl acetate. In one embodiment, a first portion of the third residue in line 234 is recycled to the second column and a second portion is purged and removed from the system via line 234. In one embodiment, once the process reaches steady state, the second portion of water to be purged is substantially similar to the amount water formed in the hydrogenation of acetic acid. In one embodiment, a portion of the third residue may be used to hydrolyze any other stream, such as one or more streams comprising ethyl acetate.

Although FIG. 5 shows third residue being directly recycled to second column 227, third residue may also be returned indirectly, for example, by storing a portion or all of the third residue in a tank (not shown) or treating the third residue to further separate any minor components such as aldehydes, higher molecular weight alcohols, or esters in one or more additional columns (not shown).

In one embodiment, a portion of the third residue in line 228 is recycled to second column 227. In one embodiment, recycling the third residue further reduces the aldehyde components in the second residue and concentrates these aldehyde components in second distillate in line 231 and thereby sent to the fourth column 235, wherein the aldehydes may be more easily separated. The third distillate in line 233 may have lower concentrations of aldehydes and esters due to the recycling of third residue in line 228.

Generally, acetic acid may be carried over in the third distillate in line 233. However, in one optional embodiment, there may be an accumulator and/or conductivity sensor for monitoring the acetic acid concentration in the third distillate in line 233.

Third column 232 is preferably a tray column as described above and operates at atmospheric pressure or optionally at pressures above or below atmospheric pressure. The temperature of the third distillate exiting in line 233 preferably is from 60° C. to 110° C., e.g., from 70° C. to 100° C. or from 75° C. to 95° C. The temperature of the third residue in line 228 preferably is from 70° C. to 115° C., e.g., from 80° C. to 110° C. or from 85° C. to 105° C. Exemplary components of the distillate and residue compositions for third column 232 are provided in Table 10 below. It should be understood that the distillate and residue may also contain other components, not listed, such as components in the feed.

THIRD COLUMN 232 (FIG. 5)

| | Conc. (wt. %) | Conc. (wt. %) | Conc. (wt. %) |
|---|---|---|---|
| Distillate | | | |
| Ethanol | 75 to 96 | 80 to 96 | 85 to 96 |
| Water | <12 | 1 to 9 | 3 to 8 |
| Acetic Acid | <12 | 0.0001 to 0.1 | 0.005 to 0.05 |
| Ethyl Acetate | <12 | 0.0001 to 0.05 | 0.005 to 0.025 |
| Acetaldehyde | <12 | 0.0001 to 0.1 | 0.005 to 0.05 |
| Diethyl Acetal | <12 | 0.0001 to 0.05 | 0.005 to 0.025 |
| Residue | | | |
| Water | 75 to 100 | 80 to 100 | 90 to 100 |
| Ethanol | <1 | 0.001 to 0.5 | 0.005 to 0.05 |
| Ethyl Acetate | <1 | 0.001 to 0.5 | 0.005 to 0.2 |
| Acetic Acid | <1 | 0.001 to 0.04 | 0.005 to 0.02 |
| Acetaldehyde | <1 | 0.0001 to 0.1 | 0.005 to 0.05 |
| Diethyl Acetal | <1 | 0.0001 to 0.05 | 0.005 to 0.025 |

In one embodiment, the third residue in line 234 is withdrawn from third column 232 at a temperature higher than the operating temperature of the second column 227. Preferably, the third residue in line 228 is integrated to heat one or more other streams or is reboiled prior to be returned to the second column 227.

On-line analyzers 215 and 218 monitor the organic impurities to reduce the buildup of those impurities in the ethanol product. Optionally, in addition to control the column parameters of the light ends column, one or more side streams from product column 232 may remove impurities. These side streams may be withdrawn in response to the impurity concentration levels detected by either of the on-line analyzers 215 and 218.

Any of the compounds that are carried through the distillation process from the feed or crude reaction product generally remain in the third distillate in amounts of less 0.1 wt. %, based on the total weight of the third distillate composition, e.g., less than 0.05 wt. % or less than 0.02 wt. %. In one embodiment, one or more side streams may remove impurities from any of the columns in the system 200. Preferably at least one side stream is used to remove impurities from the third column 232. The impurities may be purged and/or retained within the system 200.

In some embodiments, on-line analyzers and sensors 218, 219 and 220 for third column 232 may be used in combinations with on-line analyzers and sensors 213, 215, 216 and 217.

The third distillate in line 233 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit.

Returning to second column 227, the second distillate preferably is refluxed as shown in FIG. 5, optionally at a reflux ratio of 1:10 to 10:1, e.g., from 1:5 to 5:1 or from 1:3 to 3:1. The second distillate in line 231, which is a non-binary stream, may be purged or recycled to the reaction zone. In one embodiment, the second distillate in line 231 is further processed in fourth column 235, also referred to as the “acetaldehyde removal column.” In fourth column 235 the second distillate is separated into a fourth distillate, which comprises acetaldehyde, in line 236 and a fourth residue, which comprises ethyl acetate, in line 237. Each of the streams in lines 236 and 237 are non-binary streams. The fourth distillate preferably is refluxed at a reflux ratio of from 1:20 to 20:1, e.g., from 1:15 to 15:1 or from 1:10 to 10:1, and a portion of the fourth distillate is returned to the reaction zone 201. For example, the fourth distillate may be combined with the acetic acid feed, added to vaporizer 206, or added directly to the reactor 203. The fourth distillate preferably is co-fed with the acetic acid in feed line 205 to vaporizer 206. Without being bound by theory, since acetaldehyde may be hydrogenated to form ethanol, the recycling of a stream that contains acetaldehyde to the reaction zone increases the yield of ethanol and decreases byproduct and waste generation. In another embodiment, the acetaldehyde may be collected and utilized, with or without further purification, to make useful products including but not limited to n-butanol, 1,3-butanediol, and/or crotonaldehyde and derivatives.

The fourth residue of fourth column 235 may be purged via line 237. The fourth residue primarily comprises ethyl acetate and ethanol, which may be suitable for use as a solvent mixture or in the production of esters. In one preferred embodiment, the acetaldehyde is removed from the second distillate in fourth column 235 such that no detectable amount of acetaldehyde is present in the residue in line 237.

In one embodiment, a portion of the fourth residue in line 237 may be monitored using on-line analyzer 217. In response to the measured impurity concentration, the process may control a column parameter of fourth column 235. To regulate the impurity concentrations for the ethyl acetate co-product, it is preferred to control fourth column 235 instead of light ends column 227. Adjusting light ends column 227 may affect the concentration of impurities in the ethanol product. Table 11 shows the detection ranges for different components in the fourth residue. It is noted that the concentrations of the impurities in the fourth residue may be different and the column parameter may be augmented if the detected amount of the compound is outside of the detection range.

TABLE 11

| Compound | Detection Ranges |
|---|---|
| Ethanol | 0.01 wt. % - 10 wt. % |
| Acetaldehyde | 0.001 wt. % - 1.0 wt. % |
| Diethyl acetate | 0.01 wt. % - 2.0 wt. % |
| Diethyl ether | 0.001 wt. % - 0.1 wt. % |

Fourth column 235 is preferably a tray column as described above and preferably operates above atmospheric pressure. In one embodiment, the pressure is from 120 kPa to 5,000 kPa, e.g., from 200 kPa to 4,500 kPa, or from 400 kPa to 3,000 kPa. Fourth column 235 may operate at a pressure that is higher than the pressure of the other columns.

The temperature of the fourth distillate exiting in line 236 preferably is from 60° C. to 110° C., e.g., from 70° C. to 100° C. or from 75° C. to 95° C. The temperature of the residue in line 237 preferably is from 70° C. to 115° C., e.g., from 80° C. to 110° C. or from 85° C. to 110° C. Exemplary components of the distillate and residue compositions for fourth column 235 are provided in Table 12 below. It should be understood that the distillate and residue may also contain other components, not listed, such as components in the feed.

TABLE 12
FOURTH COLUMN 235 (FIG. 5)

| | Conc. (wt. %) | Conc. (wt. %) | Conc. (wt. %) |
|---|---|---|---|
| Distillate | | | |
| Acetaldehyde | 2 to 80 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Residue | | | |
| Ethyl Acetate | 40 to 100 | 50 to 100 | 60 to 100 |
| Ethanol | <40 | 0.001 to 30 | 0.01 to 15 |
| Water | <25 | 0.001 to 20 | 2 to 15 |
| Acetaldehyde | <1 | 0.001 to 0.5 | Not detectable |
| Acetal | <3 | 0.001 to 2 | 0.01 to 1 |

FIG. 6 illustrates another exemplary separation system that has a similar reaction zone 201 as FIG. 5 and produces a liquid stream 212, e.g., crude ethanol product, for further separation. FIG. 6 represents a different separation scheme than FIG. 5 that also uses sensors to determine the composition of the various streams. Because the separation scheme is different, the expected stream composition would be different than FIG. 5. In one preferred embodiment, the reaction zone 201 of FIG. 6 operates at above 70% acetic acid conversion, e.g., above 85% conversion or above 90% conversion. Thus, the acetic acid concentration in the liquid stream 212, as shown in Table 4, may be low.

Liquid stream 212 is fed to the first column 250. For purposes of convenience, the columns in each exemplary separation process, may be referred as the first, second, third, etc., columns, but it is understood that first column 250 in FIG. 6 operates differently than the first column 221 of FIG. 5. In one embodiment, no entrainers are added to first column 250. In FIG. 6, first column 250, water and unreacted acetic acid, along with any other heavy components, if present, are removed from liquid stream 212 and are withdrawn, preferably continuously, as a first residue in line 251. Preferably, a substantial portion of the water in the crude ethanol product that is fed to first column 250 may be removed in the first residue, for example, up to 75% or up to 90% of the water from the crude ethanol product. First column 250 also forms a first distillate, which is withdrawn in line 252. When column 250 is operated under about 170 kPa, the temperature of the residue exiting in line 251 preferably is from 90° C. to 130° C., e.g., from 95° C. to 120° C. The temperature of the distillate exiting in line 252 preferably is from 60° C. to 90° C., e.g., from 65° C. to 85° C. In some embodiments, the pressure of first column 250 may range from 0.1 kPa to 510 kPa.

The composition of the stream in line 251 may be determined without directly measuring the concentration of the components in the stream. In accordance to an embodiment of the invention, the composition of the binary stream in line 251 may be determined using one or more sensors 267 that measure the pressure, temperature, and/or density. The measured information may be used to infer the composition of the binary stream. When temperature-compensated density is used to infer compositional ranges for binary stream, it is preferred to use values below the maximum density. Unlike the acetic acid-water binary stream 222 in FIG. 5, the acetic acid-water binary stream in FIG. 6 is expected to contain less than 70 wt. % acetic acid. When the system detects a binary composition that indicates less acetic acid than expected, it may indicate that acetic acid is being carried over with the ethanol. The binary stream composition may be used to increase the reflux at the top of column 250 or purge the overhead stream as an off-spec product. Lower than expected concentrations of acetic acid in the binary stream may also indicate the presence of ethanol in the residue. This also is undesirable and the process may increase the reboiler temperature so that the ethanol is removed in the overhead. For purposes of clarity valves, controllers, regulators, etc., that may be used to adjust the column and/or reactor parameters are not shown in FIG. 6. The compositions of the binary stream in line 251 also may determine where the stream is directed as described above, e.g., recycled, purged, reactive distillation, neutralized, etc.

The first distillate in line 252 comprises water, in addition to ethanol and other organics. In terms of ranges, the concentration of water in the first distillate in line 252 preferably is from 4 wt. % to 38 wt. %, e.g., from 7 wt. % to 32 wt. %, or from 7 to 25 wt. %. In distillation zone 202 shown in FIG. 6, it is preferred to remove water from the first distillate in line 252. However, removing water may affect conductivity measurement. Thus, it is preferred to split first distillate 252 into two aliquot portions, in lines 253 and 254, so that the conductivity sensor measures a stream that comprises water. The relative flows into line 253 and 254 may be adjusted as necessary, but generally ranges from 10:1 to 1:10 or from 5:1 to 1:5 or 2:1 to 1:2. The portion in line 253 may be condensed and collected in an overhead accumulator 255. A conductivity sensor 256 directly monitors the conductivity of the condensed distillate in accumulator 255. In other embodiments, conductivity sensor 256 may measure a withdrawn stream from accumulator 255 as described above in FIG. 5. Conductivity sensor 256 may also be present in column 250. As discussed above, depending on the conductivity measurement, one or more column and/or reactor parameters may be adjusted such that the acetic acid levels are acceptable in the condensed distillate. For purposes of clarity valves, controllers, regulators, etc., that may be used to adjust the column and/or reactor parameters are not shown in FIG. 6.

A portion of condensed distillate in line 257 may be refluxed, for example, at a ratio of from 10:1 to 1:10, e.g., from 3:1 to 1:3 or from 1:2 to 2:1. It is understood that reflux ratios may vary with the number of stages, feed locations, column efficiency and/or feed composition. Operating with a reflux ratio of greater than 3:1 may be less preferred because more energy may be required to operate the first column 250. The remaining portion of condensed distillate in line 258 may also be fed to a second column 259.

The remaining portion of the first distillate in line 254 is fed to a water separation unit 260. Water separation unit 260 may be an adsorption unit, membrane, molecular sieves, extractive column distillation, or a combination thereof. A membrane or an array of membranes may also be employed to separate water from the distillate. The membrane or array of membranes may be selected from any suitable membrane that is capable of removing a permeate water stream from a stream that also comprises ethanol and ethyl acetate.

In a preferred embodiment, water separator 260 is a pressure swing adsorption (PSA) unit. The PSA unit is optionally operated at a temperature from 30° C. to 160° C., e.g., from 80° C. to 140° C., and a pressure of from 0.01 kPa to 550 kPa, e.g., from 1 kPa to 150 kPa. The PSA unit may comprise two to five beds. Water separator 260 may remove at least 95% of the water from the portion of first distillate in line 254, and more preferably from 99% to 99.99% of the water from the first distillate, in a water stream 261. All or a portion of water stream 261 may be returned to column 250 in line 262, where the water preferably is ultimately recovered from column 250 in the first residue in line 251. Additionally or alternatively, all or a portion of water stream 261 may be purged via line 263. The remaining portion of first distillate exits the water separator 260 as ethanol mixture stream 264. Ethanol mixture stream 264 may have a low concentration of water of less than 10 wt. %, e.g., less than 6 wt. % or less than 2 wt. %. Exemplary components of ethanol mixture stream 264 and first residue in line 251 are provided in Table 13 below. It should also be understood that these streams may also contain other components, not listed, such as components derived from the feed.

TABLE 13
FIRST COLUMN 250 WITH PSA (FIG. 6)

| | Conc. (wt. %) | Conc. (wt. %) | Conc. (wt. %) |
|---|---|---|---|
| Ethanol Mixture | | | |
| Stream | | | |
| Ethanol | 20 to 95 | 30 to 95 | 40 to 95 |
| Water | <10 | 0.01 to 6 | 0.1 to 2 |
| Ethyl Acetate | <60 | 1 to 55 | 5 to 55 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | <90 | 1 to 50 | 2 to 35 |
| Water | 30 to 100 | 45 to 95 | 60 to 90 |
| Ethanol | <1 | <0.9 | <0.3 |

In preferred embodiments, first column 250 is operated to maintain an acetic acid concentration in the first distillate of less than 700 wppm, e.g., less than 600 wppm or less than 200 wppm. In terms of ranges, the amount of acetic acid may be from 0.01 wppm to 700 wppm, e.g., from 0.1 to 600 wppm or from 0.1 to 200 wppm. In some embodiments, the first distillate may comprise substantially no acetic acid. Conductivity sensor 256 monitors the column operating conditions to maintain the acceptable operating conditions within the set baseline value. In some embodiments, when the acetic acid concentration exceeds the set baseline value, all of first distillate in line 252 is directed to accumulator 255 such that the first distillate does not contact water separation unit 260. Depending on the type of water separation unit 260, the higher acetic acid concentrations may deteriorate or impair the functions of the water separation unit 260. Thus, it may be necessary to purge the first distillate from the water separation unit 260 by collecting the first distillate in accumulator 255.

Preferably, ethanol mixture stream 264 is not returned or refluxed to first column 250. The condensed portion of the first distillate in line 258 may be combined with ethanol mixture stream 264 to control the water concentration fed to the second column 259. For example, in some embodiments the first distillate may be split into equal portions, while in other embodiments, all of the first distillate may be condensed or all of the first distillate may be processed in the water separation unit. In FIG. 6, the condensed portion in line 258 and ethanol mixture stream 264 are co-fed to second column 259. In other embodiments, the condensed portion in line 258 and ethanol mixture stream 264 may be separately fed to second column 259. The combined distillate and ethanol mixture has a total water concentration of greater than 0.5 wt. %, e.g., greater than 2 wt. % or greater than 5 wt. %. In terms of ranges, the total water concentration of the combined distillate and ethanol mixture may be from 0.5 to 15 wt. %, e.g., from 2 to 12 wt. %, or from 5 to 10 wt. %.

The second column 259 in FIG. 6, also referred to as the “light ends column,” removes ethyl acetate and acetaldehyde from the first distillate in line 258 and/or ethanol mixture stream 265. Ethyl acetate and acetaldehyde are removed as a second distillate in line 266 and ethanol is removed as the second residue in line 267. The second distillate in line 265 is a non-binary stream. The second residue in line 266 may be a binary stream comprising ethanol and water. One or more sensors 268 may measure the temperature, pressure, and/or density of second residue in line 266.

In some embodiments, the water concentration may be less than 7 wt. %, e.g., less than 4 wt. % or less than 2 wt. %. A temperature-compensated density measurement may be preferred to measure the expected higher ethanol concentrations. Based on the water concentration in line 266, the system 200 may determine if it is necessary to further dehydrate the recovered ethanol to produce a suitable ethanol product. The second residue in line 266 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit.

Second column 259 may be a tray column or packed column. In one embodiment, second column 259 is a tray column having from 5 to 70 trays, e.g., from 15 to 50 trays or from 20 to 45 trays. Second column 259 operates at a pressure ranging from 0.1 kPa to 510 kPa, e.g., from 10 kPa to 450 kPa or from 50 kPa to 350 kPa. Although the temperature of second column 259 may vary, when at about 20 kPa to 70 kPa, the temperature of the second residue exiting in line 266 preferably is from 30° C. to 75° C., e.g., from 35° C. to 70° C. The temperature of the second distillate exiting in line 265 preferably is from 20° C. to 55° C., e.g., from 25° C. to 50° C.

The total concentration of water fed to second column 259 preferably is less than 10 wt. %, as discussed above. When the condensed portion of the first distillate in line 258 and/or ethanol mixture stream 264 comprises minor amounts of water, e.g., less than 1 wt. % or less than 0.5 wt. %, additional water may be fed to the second column 259 as an extractive agent in the upper portion of the column. A sufficient amount of water is preferably added via the extractive agent such that the total concentration of water fed to second column 259 is from 1 wt. % to 10 wt. % water, e.g., from 2 to 6 wt. %, based on the total weight of all components fed to second column 259. If the extractive agent comprises water, the water may be obtained from an external source or from an internal return/recycle line from one or more of the other columns or water separators.

Suitable extractive agents are discussed herein, and include water. When extractive agents are used, a suitable recovery system, such as a further distillation column, may be used to recycle the extractive agent.

Exemplary components for the second distillate and second residue compositions for the second column 259 are provided in Table 14, below. It should be understood that the distillate and residue may also contain other components, not listed in Table 14.

TABLE 14
SECOND COLUMN 259 (FIG. 6)

| | Conc. (wt. %) | Conc. (wt. %) | Conc. (wt. %) |
|---|---|---|---|
| Second Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| Second Residue | | | |
| Ethanol | 80 to 99.5 | 85 to 99.5 | 90 to 99.5 |
| Water | <20 | 0.001 to 15 | 0.01 to 10 |
| Ethyl Acetate | <1 | 0.001 to 2 | 0.001 to 0.5 |
| Acetic Acid | <0.5 | <0.01 | 0.001 to 0.01 |

The second distillate in line 265, which comprises ethyl acetate and/or acetaldehyde, preferably is refluxed as shown in FIG. 6, for example, at a reflux ratio of from 1:30 to 30:1, e.g., from 1:10 to 10:1 or from 1:3 to 3:1. In one aspect, not shown, the second distillate 265 or a portion thereof may be returned to reactor 203.

In one embodiment, the second distillate in line 265 may be further separated to produce an acetaldehyde-containing stream and an ethyl acetate-containing stream using a fourth column described above in FIG. 5. This may allow a portion of either the resulting acetaldehyde-containing stream or ethyl acetate-containing stream to be recycled to reactor 203 while purging the other stream. The purge stream may be valuable as a source of either ethyl acetate and/or acetaldehyde.

FIG. 7 illustrates different exemplary separation system scheme than FIGS. 5 and 6 that also uses on-line analyzers to produce ethanol. Because the separation scheme is different, the expected stream composition would be different than FIGS. 5 and 6. However, similar to FIGS. 5 and 6, the concentration of some streams may be determined without actually measuring the concentration of the streams. For binary streams that typically contain two components, their concentration may be inferred by measuring the temperature, pressure, and density of the streams. Reaction zone 201 of FIG. 7 is similar to FIGS. 5 and 6 and produces a liquid stream 212, e.g., crude ethanol product, for further separation. In one preferred embodiment, the reaction zone 201 of FIG. 7 operates at above 80% acetic acid conversion, e.g., above 90% conversion or above 99% conversion. Thus, the acetic acid concentration in the liquid stream 212 may be low.

In the exemplary embodiment shown in FIG. 7, liquid stream 212 is introduced in the lower part of first column 270, e.g., lower half or middle third. In one embodiment, no entrainers are added to first column 270. In first column 270, a weight majority of the ethanol, water, acetic acid, and other heavy components, if present, are removed from liquid stream 212 and are withdrawn, preferably continuously, as residue in line 271. First column 270 also forms an overhead distillate, which is withdrawn in line 272, and which may be condensed and refluxed, for example, at a ratio of from 30:1 to 1:30, e.g., from 10:1 to 1:10 or from 1:5 to 5:1. The overhead distillate in stream 272 preferably comprises a weight majority of the ethyl acetate from liquid stream 212. The distillate in line 272 and residue in line 271 preferably are non-binary streams and no sensors are used to infer the composition of these streams.

When column 270 is operated under about 170 kPa, the temperature of the residue exiting in line 271 preferably is from 70° C. to 155° C., e.g., from 90° C. to 130° C. or from 100° C. to 110° C. The base of column 270 may be maintained at a relatively low temperature by withdrawing a residue stream comprising ethanol, water, and acetic acid, thereby providing an energy efficiency advantage. The temperature of the distillate exiting in line 272 preferably at 170 kPa is from 75° C. to 100° C., e.g., from 75° C. to 83° C. or from 81° C. to 84° C. In some embodiments, the pressure of first column 270 may range from 0.1 kPa to 510 kPa. Exemplary components of the distillate and residue compositions for first column 270 are provided in Table 15 below. It should also be understood that the distillate and residue may also contain other components, not listed in Table 15.

TABLE 15
FIRST COLUMN 270 (FIG. 7)

| | Conc. (wt. %) | Conc. (wt. %) | Conc. (wt. %) |
|---|---|---|---|
| Distillate | | | |
| Ethyl Acetate | 10 to 85 | 15 to 80 | 20 to 75 |
| Acetaldehyde | 0.1 to 70 | 0.2 to 65 | 0.5 to 65 |
| Acetal | <0.1 | <0.1 | <0.05 |
| Acetone | <0.05 | 0.001 to 0.03 | 0.01 to 0.025 |
| Ethanol | 3 to 55 | 4 to 50 | 5 to 45 |
| Water | 0.1 to 20 | 1 to 15 | 2 to 10 |
| Acetic Acid | <2 | <0.1 | <0.05 |
| Residue | | | |
| Acetic Acid | 0.01 to 35 | 0.1 to 30 | 0.2 to 25 |
| Water | 25 to 70 | 30 to 65 | 35 to 60 |
| Ethanol | 10 to 75 | 15 to 70 | 20 to 65 |

In an embodiment of the present invention, column 270 may be operated at a temperature where most of the water, ethanol, and acetic acid are removed from the residue stream and only a small amount of ethanol and water is collected in the distillate stream due to the formation of binary and tertiary azeotropes. The weight ratio of water in the residue in line 271 to water in the distillate in line 272 may be greater than 1:1, e.g., greater than 2:1. The weight ratio of ethanol in the residue to ethanol in the distillate may be greater than 1:1, e.g., greater than 2:1.

The distillate in line 272 preferably is substantially free of acetic acid that comprises less than 1000 wppm acetic acid, e.g. less than 500 wppm or less than 100 wppm. In some optional embodiments, a conductivity sensor may measure the acetic acid concentration in the first distillate in line 272. However, because a majority of the ethanol and acetic acid are removed together in the first residue in line 271, it may be preferred to monitor acetic acid concentrations in the second distillate as shown in FIG. 7.

The distillate in line 272 may be purged from the system or recycled in whole or part to reactor 203. In some embodiments, the distillate may be further separated, e.g., in a distillation column (not shown), into an acetaldehyde stream and an ethyl acetate stream. Either of these streams may be returned to the reactor 203 or separated from system 200 as a separate product.

The amount of acetic acid in the first residue may vary depending primarily on the conversion in reactor 203. In one embodiment, when the conversion is high, e.g., greater than 90%, the amount of acetic acid in the first residue may be less than 10 wt. %, e.g., less than 5 wt. % or less than 2 wt. %. In other embodiments, when the conversion is lower, e.g., less than 90%, the amount of acetic acid in the first residue may be greater than 10 wt. %.

To recover ethanol, the residue in line 271 may be further separated in a second column 273, also referred to as an “acid separation column.” An acid separation column may be used when the acetic acid concentration in the first residue is greater than 1 wt. %, e.g., greater than 5 wt. %. The first residue in line 271 is preferably introduced in the top part of column 273, e.g., top half or top third. Second column 273 yields a second residue in line 274 comprising acetic acid and water, and a second distillate in line 275 comprising ethanol. A portion of the second distillate may be refluxed at a ratio of from 10:1 to 1:10, e.g., from 3:1 to 1:3 or from 1:2 to 2:1.

The second residue in line 274 may be an acetic acid-water binary stream. One or more sensors 281 may be used to monitor the temperature, pressure, and/or density of line 274 and infer a binary composition. The second distillate in line 275 may be an ethanol-water binary stream. One or more sensors 280 may be used to monitor the temperature, pressure, and/or density of line 275 and infer a binary composition. As discussed above, depending on the inferred binary stream composition, one or more column and/or reactor parameters may be adjust such that the acetic acid levels are acceptable in the second distillate. For purposes of clarity valves, controllers, regulators, etc., that may be used to adjust the column and/or reactor parameters are not shown in FIG. 7.

Second distillate, which is withdrawn in line 275 is condensed and collected in an overhead accumulator 276. As shown in FIG. 7, a conductivity sensor 277 directly monitors the conductivity of the condensed distillate in accumulator 276. The condensed distillate in accumulator 276 comprises primarily ethanol, with azeotropic amounts of water, and low amounts of other organic compounds. Due to the low amounts of water, the dissociation of acetic acid in accumulator 276 may be low and embodiments may require a sensitive conductivity sensor 277. In particular, the conductivity sensor should measure values below 0.1 micromhos/cm. In other embodiments, conductivity sensor 277 may measure a withdrawn stream from accumulator 276 as described above in FIG. 1. As discussed above, depending on the conductivity measurement, one or more column and/or reactor parameters may be adjust such that the acetic acid levels are acceptable in the condensed distillate. For purposes of clarity valves, controllers, regulators, etc., that may be used to adjust the column and/or reactor parameters are not shown in FIG. 7. A portion of the condensed distillate may be refluxed via line 278 at a ratio of from 10:1 to 1:10, e.g., from 3:1 to 1:3 or from 1:2 to 2:1. The remaining portion of the condensed distillate is withdrawn via line 279 as the ethanol product. Because ethanol is withdrawn as a product from accumulator 276 it may be more preferred to closely monitor the acetic acid concentrations with sensor 277 and set lower acceptable baseline levels. To produce an ethanol there is a lower tolerance of higher acetic acid concentration.

Second column 273 may be a tray column or packed column. In one embodiment, second column 273 is a tray column having from 5 to 150 trays, e.g., from 15 to 50 trays or from 20 to 45 trays. Although the temperature and pressure of second column 273 may vary, when at atmospheric pressure the temperature of the second residue exiting in line 274 preferably is from 95° C. to 130° C., e.g., from 100° C. to 125° C. The temperature of the second distillate exiting in line 275 preferably is from 60° C. to 105° C., e.g., from 75° C. to 100° C. The pressure of second column 273 may range from 0.1 kPa to 510 kPa. Exemplary components for the distillate and residue compositions for second column 273 are provided in Table 16 below. It should be understood that the distillate and residue may also contain other components, not listed in Table 16.

TABLE 16
SECOND COLUMN 273 (FIG. 7)

| | Conc. (wt. %) | Conc. (wt. %) | Conc. (wt. %) |
|---|---|---|---|
| Second Distillate | | | |
| Ethanol | 70 to 99.9 | 75 to 98 | 80 to 95 |
| Ethyl Acetate | <10 | 0.001 to 5 | 0.01 to 3 |
| Acetaldehyde | <5 | 0.001 to 1 | 0.005 to 0.5 |
| Water | 0.1 to 30 | 1 to 25 | 5 to 20 |
| Second Residue | | | |
| Acetic Acid | 0.1 to 45 | 0.2 to 40 | 0.5 to 35 |
| Water | 45 to 100 | 55 to 99.8 | 65 to 99.5 |
| Ethyl Acetate | <2 | <1 | <0.5 |
| Ethanol | <5 | 0.001 to 5 | <2 |

The weight ratio of ethanol in the second distillate in line 275 to ethanol in the second residue in line 274 preferably is at least 35:1. In one embodiment, the weight ratio of water in the second residue 274 to water in the second distillate 275 is greater than 2:1, e.g., greater than 4:1 or greater than 6:1. In addition, the weight ratio of acetic acid in the second residue 274 to acetic acid in the second distillate 275 preferably is greater than 10:1, e.g., greater than 15:1 or greater than 20:1. Preferably, the second distillate in line 275 is substantially free of acetic acid and may only contain, if any, trace amounts of acetic acid. Preferably, the second distillate in line 275 contains substantially no ethyl acetate.

The remaining water from the second distillate withdrawn in line 279 may be removed in further embodiments of the present invention. Depending on the water concentration, the ethanol product may be derived from the second distillate. Some applications, such as industrial ethanol applications, may tolerate water in the ethanol product, while other applications, such as fuel applications, may require an anhydrous ethanol. The amount of water in the second distillate withdrawn in line 279 may be closer to the azeotropic amount of water, e.g., at least 4 wt. %, preferably less than 20 wt. %, e.g., less than 12 wt. % or less than 7.5 wt. %. Water may be removed from the second distillate using several different separation techniques as described herein. Particularly preferred techniques include the use of distillation column, membranes, adsorption units, and combinations thereof.

Some of the residues withdrawn from the separation zone comprise acetic acid and water. Depending on the amount of water and acetic acid, the residue may be treated in one or more of the following processes. The following are exemplary processes for further treating the residue and it should be understood that any of the following may be used regardless of acetic acid concentration. When the residue comprises a majority of acetic acid, e.g., greater than 70 wt. %, the residue may be recycled to the reactor without any separation of the water. In one embodiment, the residue may be separated into an acetic acid stream and a water stream when the residue comprises a majority of acetic acid, e.g., greater than 50 wt. %. Acetic acid may also be recovered in some embodiments from the residue having a lower acetic acid concentration. The residue may be separated into the acetic acid and water streams by a distillation column or one or more membranes. If a membrane or an array of membranes is employed to separate the acetic acid from the water, the membrane or array of membranes may be selected from any suitable acid resistant membrane that is capable of removing a permeate water stream. The resulting acetic acid stream optionally is returned to the hydrogenation reactor. The resulting water stream may be used as an extractive agent or to hydrolyze an ester-containing stream in a hydrolysis unit.

In other embodiments, for example, where the residue comprises less than 50 wt. % acetic acid, possible options include one or more of: (i) returning a portion of the residue to hydrogenation reactor, (ii) neutralizing the acetic acid, (iii) reacting the acetic acid with an alcohol, or (iv) disposing of the residue in a waste water treatment facility. It also may be possible to separate a residue comprising less than 50 wt. % acetic acid using a weak acid recovery distillation column to which a solvent (optionally acting as an azeotroping agent) may be added. Exemplary solvents that may be suitable for this purpose include ethyl acetate, propyl acetate, isopropyl acetate, butyl acetate, vinyl acetate, diisopropyl ether, carbon disulfide, tetrahydrofuran, isopropanol, ethanol, and C₃-C₁₂ alkanes. When neutralizing the acetic acid, it is preferred that the residue comprises less than 10 wt. % acetic acid. Acetic acid may be neutralized with any suitable alkali or alkaline earth metal base, such as sodium hydroxide or potassium hydroxide. When reacting acetic acid with an alcohol, it is preferred that the residue comprises less than 50 wt. % acetic acid. The alcohol may be any suitable alcohol, such as methanol, ethanol, propanol, butanol, or mixtures thereof. The reaction forms an ester that may be integrated with other systems, such as carbonylation production or an ester production process. Preferably, the alcohol comprises ethanol and the resulting ester comprises ethyl acetate. Optionally, the resulting ester may be fed to the hydrogenation reactor.

In some embodiments, when the residue comprises very minor amounts of acetic acid, e.g., less than 5 wt. %, the residue may be disposed of to a waste water treatment facility without further processing. The organic content, e.g., acetic acid content, of the residue beneficially may be suitable to feed microorganisms used in a waste water treatment facility.

Returning to first column 270, a first distillate in line 272 is withdrawn therefrom. A portion of the first distillate may be refluxed at a ratio of from 10:1 to 1:10, e.g., from 3:1 to 1:3 or from 1:2 to 2:1. The first distillate in line 272 comprises primarily ethanol, as well as water, ethyl acetate, acetaldehyde, and/or diethyl acetal. The first distillate is a non-binary stream. Acetic acid concentration in the first distillate preferably is from 10 to 1000 wppm, e.g., from 50 to 600 wppm, or from 100 to 200 wppm. The compositions of the binary streams in the process may be monitored to control the acetic acid concentration in the first distillate. Carrying larger amounts of acetic acid with the recovered ethanol may lead to further inefficiencies and costs.

The columns shown in figures may comprise any distillation column capable of performing the desired separation and/or purification. For example, other than the acid columns describe above, the other columns preferably are a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these zones although in some embodiments subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The final ethanol product produced by the processes of the present invention may be taken from a stream that primarily comprises ethanol. The ethanol product may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 17.

TABLE 17
FINISHED ETHANOL COMPOSITIONS

| Component | Conc. (wt. %) | Conc. (wt. %) | Conc. (wt. %) |
|---|---|---|---|
| Ethanol | 75 to 99.9 | 80 to 99.5 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit as discussed above. In such embodiments, the ethanol concentration of the ethanol product may be greater than indicated in Table 11, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated herein by reference.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising:
hydrogenating an acetic acid feed stream in the presence of a catalyst to form a crude ethanol product;
separating the crude ethanol product in one or more distillation columns, wherein acetic acid is present in a residue and ethanol is present in a distillate, and wherein at least one of the distillate or residue comprises an organic impurity;
measuring temperature, pressure, density, concentration, or conductivity of at least one of the distillate or the residue from the one or more distillation columns;
setting a baseline value for the at least one of the distillate or the residue;
adjusting at least one column parameter based on the measured data and the baseline value; and
recovering the ethanol product.

2. The process of claim 1, wherein the measuring is conducted using one or more on-line analyzers.

3. The process of claim 2, wherein the on-line analyzer is selected from the group consisting of gas chromatograph, Raman spectrometer, high-performance liquid chromatograph, mass spectrometer, infrared spectrometer, and near-infrared spectrometer.

4. The process of claim 1, wherein the at least one column parameter is selected from the group consisting of reflux ratio, residue to feed ratio, distillate to feed ratio, column temperature, column pressure, reboiler energy input, and combinations thereof.

5. The process of claim 1, wherein the organic impurity is selected from the group consisting of ethyl acetate, acetaldehyde, diethyl acetal, diethyl ether, and C₃-C₄ alcohols.

6. The process of claim 5, wherein the concentration of ethyl acetate is between 10 wppm to 100 wppm.

7. The process of claim 5, wherein the concentration of acetaldehyde is between 10 wppm to 100 wppm.

8. The process of claim 5, wherein the concentration of diethyl acetal is between 10 wppm to 100 wppm.

9. The process of claim 5, wherein the concentration of diethyl ether is between 10 wppm to 100 wppm.

10. The process of claim 5, wherein the concentration of C₃-C₄ alcohols is between 11 wppm to 220 wppm.

11. The process of claim 1, wherein the baseline value is a conductivity value of less than 1000 wppm.

12. The process of claim 1, wherein the residue comprises water and acetic acid.

13. The process of claim 1, wherein the distillate comprises water and ethanol.

14. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil petroleum, coal, biomass, and combinations thereof.

15. A process for effecting process control in a distillation column for separating a reaction mixture produced by hydrogenating acetic acid to form ethanol, wherein the reaction mixture comprises ethanol and acetic acid, the process comprising the steps of:
measuring a conductivity of a condensed distillate of the distillation column, wherein the condensed distillate comprises ethanol and acetic acid;
controlling at least one parameter of the distillation column in response to the measured conductivity;
setting a baseline conductivity value for the condensed distillate and comparing the measured conductivity with the baseline conductivity value;
returning the condensed distillate to the distillation column based on the measured conductivity; and
recovering ethanol from the condensed distillate.

16. The process of claim 15, wherein the baseline conductivity value represents an acetic acid concentration less than 1000 wppm.

17. A process for producing ethanol, comprising:
hydrogenating an acetic acid feed stream in the presence of a catalyst to form a crude ethanol product;
separating at least a portion of the crude ethanol product in a first column into a first distillate comprising ethanol, water, and ethyl acetate, and a first residue comprising acetic acid;
separating at least a portion of the first distillate in a second column into a second distillate comprising ethyl acetate, and a second residue comprising ethanol, water, and at least one organic impurity;
separating at least a portion of the second residue in a third column into a third distillate comprising ethanol and a third residue comprising water;
measuring the concentration of the at least one organic impurity using one or more on-line analyzers; and
adjusting at least one column parameter of the second column to maintain the organic impurity concentration below 1 wt. %.

18. The process of claim 17, wherein the at least one column parameter is selected from the group consisting of reflux ratio, residue to feed ratio, distillate to feed ratio, column temperature, column pressure, reboiler energy input, and combinations thereof.

19. The process of claim 17, wherein the on-line analyzer is selected from the group consisting of gas chromatograph, Raman spectrometer, high-performance liquid chromatograph, mass spectrometer, infrared spectrometer, and near-infrared spectrometer.

20. The process of claim 17, wherein the one column parameter is selected from the group consisting of reflux ratio, residue to feed ratio, distillate to feed ratio, column temperature, column pressure, reboiler energy input, and combinations thereof.
